(19) United States
(12) Patent Application Publication
(10) Pub. No.: US 2012/0196918 A1
(43) Pub. Date: Aug. 2, 2012

(54) PREVENTING ISLET INFLAMMATION AND DYSFUNCTION AND MAINTAINING PROPER GLUCOSE LEVELS BY CONTROLLING eIF5A AND ITS HYPUSINATION

(76) Inventors: Raghavendra G. Mirmira, Zionsville, IN (US); Bernhard Maier, Carmel, IN (US)

(21) Appl. No.: 13/263,610

(22) PCT Filed: Apr. 8, 2010
(86) PCT No.: PCT/US2010/030379

§ 371 Date: Feb. 17, 2012

Related U.S. Application Data
(60) Provisional application No. 61/167,701, filed on Apr. 8, 2009.

(51) Int. Cl. A61K 31/7088; A61P 3/10; A61K 31/167; A61K 31/155; A61K 31/4412

(52) U.S. Cl. 514/44 A; 514/634; 514/348; 514/614

(57) ABSTRACT

Pancreatic islet dysfunction, in both type 1 and type 2 diabetes results, in part, from cytokine-mediated inflammation leading to iNOS generation and the death of pancreatic islets. The production of pro-inflammatory cytokines involved in the generation of iNOS is facilitated by the availability of the hypusine-containing translational factor eIF5A, necessary for the maturation of antigen-presenting cells. Treatment with agents capable of interfering with the mRNA translating iNOS or with agents that can interfere with the hypusination of eIF5A, prevents the death of islets, lowers blood glucose levels, avoids insulin resistance, and generally avoids the inflammatory response in islets associated with type 1 and type 2 diabetes.

### CROSS-REFERENCES TO RELATED APPLICATIONS

This application is a National Stage filing of International Application PCT/US10/30379, filed Apr. 8, 2010, claiming priority to U.S. Provisional Application No. 61/167,701, filed Apr. 8, 2009, entitled “PREVENTING ISLET INFLAMMATION AND DYSFUNCTION AND MAINTAINING PROPER GLUCOSE LEVELS BY CONTROLLING eIF5A AND ITS HYPUSINATION.” The subject application claims priority to PCT/US10/30379, and to U.S. Provisional Application No. 61/167,701, and incorporates all by reference herein, in their entirety.

### STATEMENT REGARDING FEDERALLY SPONSORED RESEARCH

This invention was made with government support under DK060581 awarded by the National Institutes of Health. The U.S. Government has certain rights in the invention.

### STATEMENT REGARDING NO-FEDERALLY SPONSORED RESEARCH

This work was additionally supported, in part, by an investigator-initiated grant from Senesco Technologies, Inc.,

### BACKGROUND

Diabetes is a disorder of glucose homeostasis that affects over 200 million people world-wide. Dysfunction or destruction of islet β cells appears to underlie all forms of diabetes. Whereas type 1 diabetes results from the auto-immune destruction of islet β cells, type 2 diabetes is thought to develop as β cell insulin release is unable to compensate for an increasing insulin demand (1). Emerging data suggest that in both forms of diabetes the release of pro-inflammatory cytokines is central to triggering pathways that initiate β cell dysfunction and eventual cell death. In the case of type 1 diabetes, a complex interplay between β cells and cells of the immune system leads to the recruitment of activated CD4+ T cells and macrophages to the vicinity of the islet, resulting in local release of pro-inflammatory cytokines (IL-1β, TNFα, and IFNγ)

(2). In the case of type 2 diabetes, systemic insulin resistance leads to increased circulating pro-inflammatory cytokines (3), whereas exogenous administration of IL-1 receptor antagonist (IL-1Ra) has been demonstrated to reduce glycemia and improve β cell function in mice with diet-induced hyperglycemia (4) and human subjects with type 2 diabetes (5).

Pro-inflammatory cytokines acutely trigger NFκB-mediated transcription of the Nos2 gene encoding inducible nitric oxide synthase (6). Production of nitric oxide by iNOS contributes to the early pathogenesis of β cell dysfunction in response to cytokines, as nitric oxide inhibits proteins involved in aerobic glycolysis and the electron transport chain, thereby diminishing cellular ATP production (7). This impairment in ATP production limits the coupling of glycolysis to insulin release in the β cell (8). In the longer term, both the iNOS-dependent and independent effects of cytokine signaling lead to eventual islet death (9, 10, 11, and 12). Thus, to preserve islet function in the setting of inflammation, it is imperative to identify and counter the mechanisms that mediate islet responsiveness to pro-inflammatory cytokines.

Eukaryotic translation initiation factor 5A (eIF5A) is a small (17 kDa) acidic protein that is highly conserved throughout evolution (13); eIF5A is the only protein known to contain the unique polyamine-derived amino acid hypusine (N^{ε}-(4-amino-2-hydroxybutyl)-lysine) (14). Hypusine is formed posttranslationally during a reaction involving residue Lys50 of eIF5A and the enzymes deoxyhypusine synthase (DHS) and deoxyhypusine hydroxylase (DOHH), and is necessary for many eIF5A functions (for review see ref 15). In mammalian cells, eIF5A appears to be a mediator of cellular proliferation (16 and 17) and apoptosis (18, 19, and 20), but its mechanisms have remained vague. The administration of small interfering (si)RNA against eIF5A to mice significantly reduced endotoxin (lipopolysaccharide)-induced lethality as well as suppressed the production of IL-1, TNFα, and chemokines in the lungs following endotoxin challenge (21). Taken together, these studies suggest that eIF5A participates in and can be essential to inflammatory responses.

The role of eIF5A in the pathogenesis of islet dysfunction in diabetes has not been directly examined. In the non-obese diabetic (NOD) mouse model of type 1 diabetes, approximately 30 distinct chromosomal loci have been identified that appear to contribute to the susceptibility of diabetes (known as “Idd” loci) (22). Interestingly, one of these loci on the distal arm of chromosome 11 (Idd4) harbors genes that are seminal to the autoimmune inflammatory response (e.g. IL12b, Trpv1, Nos2, Alox15) (23), and includes the gene encoding eIF5A. In the context of autoimmunity and inflammation, studies of Hauber and colleagues (24) demonstrated that the hypusinated form of eIF5A (eIF5A-Hyp) is essential for the expression of CD83, a cell surface marker that correlates with the maturation of antigen presenting cells. Thus, eIF5A-Hyp appears to be important in the early pathogenesis of the immune response in autoimmune diseases such as type 1 diabetes. However, because pancreatic islets express eIF5A, the present study considers the possibility that eIF5A participates in the islet response to autoimmunity and inflammation. The present study demonstrates that eIF5A-Hyp enables cytokine-mediated islet dysfunction through the direct post-transcriptional regulation of the mRNA encoding iNOS (Nos2) in both rodent and human cells. Further, the study shows that depletion of eIF5A or inhibition of hypusination can protect against the development of glucose intolerance in inflammatory mouse models of diabetes. These findings point to a novel pathway in which cytokines are linked to iNOS production via the post-transcriptional regulation of Nos2 by eIF5A-Hyp. These studies have demonstrated that targeting of hypusination represents a therapeutic strategy to mitigate the inflammatory response in pancreatic islets.

To assist the reader the following listing of non-standard abbreviations used herein are provided:

eIF5A, eukaryotic translation initiation factor 5A;
DHS, deoxyhypusine synthase;
DOHH, deoxyhypusine hydroxylase;
GSCa, glucose-stimulated Ca²⁺ mobilization;
GSIS, glucose-stimulated insulin secretion;
eIF5A-Hyp, hypusinated eIF5A
IL-1Ra, interleukin-1 receptor antagonist
iNOS, inducible nitric oxide synthase;
IPGTT, intraperitoneal glucose tolerance test;
LPS, lipopolysaccharide
siRNA, small interfering RNA; and
STZ, streptozotocin.

### SUMMARY

The hypusine-containing protein eIF5A is necessary for the maturation of antigen-presenting cells and facilitates pro-inflammatory cytokine production by immune cells. The protein, eIF5A is also expressed in pancreatic islets, and has now been shown to promote the inflammatory response in islets during the development of diabetes. To demonstrate this, eIF5A was depleted in mice by RNA interference and the observation made that animals were resistant to β cell degranulation and the development of hyperglycemia in the low dose streptozotocin diabetes model. The protection afforded by eIF5A depletion resulted from impaired translation of the mRNA encoding the inflammatory enzyme inducible nitric oxide synthase (iNOS) within the islet. In rodent β cells and human islets in vitro, cytokine-induced iNOS translation was dose-dependently reduced in the presence of inhibitors of hypusine synthesis, indicating a role for the hypusine residue in mediating islet inflammation. It has also been demonstrated that hypusine is required in part for the nuclear-to-cytoplasmic transport of iNOS mRNA, and that this transport process involves interactions between hypusinated eIF5A, iNOS mRNA, and the export protein exportin1/CRM1. Mice treated with an inhibitor of hypusination displayed resistance to streptozotocin diabetes and a block in iNOS production in islets.

A first aspect of the present disclosure involves an in vivo method for treating a condition or disease selected from the group including insulin resistance, an elevated blood glucose level, pre-diabetes, diabetes 1, and diabetes 2. The method includes the steps of providing a mammal exhibiting symptoms of said condition or disease; and treating said mammal with a therapeutically effective amount of an agent capable of blocking or attenuating iNOS translation within said mammal's pancreatic islets. The method is particularly suitable for treating humans. Agents utilized are formulated in a pharmaceutically acceptable agent.

The translation of iNOS can be effected with treatment of a siRNA, an inhibitor of deoxyhypusine synthase, or an inhibitor of deoxyhypusine synthase. Treatment with a siRNA can involve treatment with si-eIF5A. Treatment with an inhibitor of deoxyhypusine synthase can involve treatment with GC6, GC7, GC8, GC6G, GC7G, GC8G, CN-1493, and a combination thereof. Treatment with GC7 is preferred. Finally, treatment with an inhibitor of deoxyhypusine hydroxylase can involve treatment with mimosine.

A further aspect of the present disclosure involves an in vivo method for controlling a mammal's blood glucose level. The method involves providing a mammal exhibiting an elevated blood glucose level and treating the mammal with an effective amount of an agent formulated in a pharmaceutically acceptable carrier and capable of reducing iNOS production within the mammal's pancreatic islets. Treating the mammal in this manner lowers the mammal's blood glucose level below the initial elevated blood glucose level.

The translation of iNOS can be effected with treatment of a siRNA, an inhibitor of deoxyhypusine synthase, or an inhibitor of deoxyhypusine hydroxylase. Treatment with a siRNA can involve treatment with si-eIF5A. Treatment with an inhibitor of deoxyhypusine synthase can involve treatment with GC6, GC7, GC8, GC6G, GC7G, GC8G, CNI-1493, and a combination thereof. Treatment with GC7 is preferred. Finally, treatment with an inhibitor of deoxyhypusine hydroxylase can involve treatment with mimosine.

A still further aspect of the present disclosure involves an in vivo method for treating a condition or disease that can include insulin resistance, an elevated blood glucose level, pre-diabetes, diabetes 1, and diabetes 2 by providing a mammal exhibiting symptoms of the condition or disease; and treating the mammal with a therapeutically effective amount of an agent capable of inhibiting hypusination of eIF5A within the mammal's pancreatic islets. Agents are typically formulated in a pharmaceutically acceptable carrier.

The translation of iNOS can be effected with treatment of a siRNA, an inhibitor of deoxyhypusine synthase, or an inhibitor of deoxyhypusine synthase. Treatment with a siRNA can involve treatment with si-eIF5A. Treatment with an inhibitor of deoxyhypusine synthase can involve treatment with GC6, GC7, GCB, GC6G, GC7G, GC8G, CNI-1493, and a combination thereof. Treatment with GC7 is preferred. Finally, treatment with an inhibitor of deoxyhypusine hydroxylase can involve treatment with mimosine.

As used herein, the term “pharmaceutically acceptable carriers” means a non-toxic, inert solid, semi-solid or liquid filler, diluent, encapsulating material or formulation auxiliary of any type. Some examples of the materials that can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; carboxylic acids such as acetic acid, buffering agents such as magnesium hydroxide and aluminum hydroxide; alginic acid; pyrogen-free water; isotonic saline; Ringer's solution, ethyl alcohol and phosphate buffer solutions, as well as other non-toxic compatible substances used in pharmaceutical formulations. Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgement of the formulator. Examples of pharmaceutically acceptable. antioxidants include—water soluble antioxidants such as ascorbic acid, cysteine hydrochloride, sodium bisulfite, sodium metabisulfite, sodium sulfite, and the like; oil soluble antioxidants such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

By use of the “term effective amount” includes a sufficient amount of the agent to elicit the desired result, i.e., the desired pharmacological or biochemical result over the amount in which no result is observed. Preferred embodiments are described below.

The following suggested dosages are meant to be illustrative and are not meant to be limiting. For each of the methods described above, preferred dosage for the agent depends on the method of application and the level of toxicity, if any, that can be tolerated. For example a single dose treatment such as an injection of GC7, an oral dosage, and the like, treatments can range from about 0.1 mg/kg/day to about 10 mg/kg/day, more preferably from about 0.3 mg/kg/day to about 5 mg/kg/day, and most preferably from about 1 mg/kg/day to about 4 mg/kg/day. For continuous or semi-continuous application such as delivery of GC7 through a pump or saline drip, treatments can range from about 1 mg/kg/day to about 40 mg/kg/day, more preferably from about 2 mg/kg/day to about 10 mg/kg/day, and still more preferably from about 3 mg/kg/day to about 6 mg/kg/day. Higher dosages of an agent selected can be utilized and preferred provided the agent has minimal toxic side effects. Toxic side effects have not been experienced with the dosages studied at this time. With this disclosure in hand, one skilled in the art can readily optimize the appropriate dosages for any of the agents taught.

A still further aspect of the present disclosure involves a composition for treating a condition or disease that can include insulin resistance, an elevated blood glucose level, pre-diabetes, diabetes 1, and diabetes 2. Treatment involves the administration of an agent capable of inhibiting iNOS translation within a pancreatic cell included in a pharmaceutically acceptable carrier. Suitable agents can be selected from the group consisting of si-eIF5A, GC6, GC7, GC8, GC6G, GC7G, GC8G, and a combination thereof. A preferred si-eIF5A includes a nucleotide having the sequence 5′-AACGGAAUGACUUCCAGCUGA-3 (SEQ ID NO: 2). A preferred inhibitor of hypusination includes GC7. The concentration of the agent in the carrier typically ranges from about 0.1 μM to about 200 μM, preferably from about 0.3 μM to about 125 μM, more preferably from about 1 μM to about 100 μM, still more preferably from about 2 μM to about 30 μM, and finally most preferably from about 3 μM to about 10 μM.

Finally, a further aspect of the current disclosure includes an agent capable of reducing iNOS production within pancreatic islets for use in the treatment of diabetes. Suitable agents include siRNA's and inhibitors of the hypusination of eIF5A. Suitable siRNA's include si-eIF5A and suitable inhibitors of the hypusination of iIF5A include inhibitors of deoxyhypusine synthase and inhibitors of deoxyhypusine hydroxylase. A suitable si-eIF5A includes the nucleotide sequence 5′-AACGGAAUGACUUCCAGCUGA-3 (SEQ ID NO: 2). Examples of inhibitors of deoxyhypusine synthase include GC6, GC7, GC8, GC6G, GC7G, GC8G, CNI-1493, and combinations thereof. GC7 is a particularly effective inhibitor. Examples of inhibitors of deoxyhypusine hydroxylase include mimosine.

### FIGURES

FIG. 1A illustrates a schematic of the STZ, IL-1Ra, and siRNA injection protocol utilized in immunocompetent mice.

FIG. 1B illustrates the results of intraperitoneal GTTs at day 7 in C57BL/6J male mice.

FIG. 1C illustrates the results of intraperitoneal GTTs at day 7 in NOD/Scid-(IL-2Rg-null)male mice.

FIG. 1D illustrates scatter plot showing individual fasting blood glucoses of untreated C57BL/6J male mice, or STZ-treated mice injected with si-Control or si-eIF5A at day 7.

FIG. 1E illustrates a plot of intraperitoneal GTT's at day 7 for untreated C57BL/6J male mice, or STZ-treated mice injected with si-Control or si-eIF5A at day 7.

FIG. 2A illustrates pancreata from untreated mice and STZ-treated mice injected with si-Control or si-eIF5A at low (upper panels) and high (lower panels) magnification.

FIG. 2B illustrates a β cell mass in untreated C57BL/6J mice and STZ-treated mice injected with si-Control and si-eIF5A.

FIG. 2C illustrates pancreata from mice at the end of the study were paraffin-embedded and stained for iNOS and counterstained with hematoxylin.

FIG. 2D illustrates immunoblots of islet extract from untreated mice and from si-Control- and si-eIF5A-injected mice following a single dose of STZ.

FIG. 3A illustrates representative immunoblot of islet extract for actin, eIF5A, and si-Control- and si-eIF5A-treated animals that were subjected to a 4 hour pulse of ³H-spermidine after the extract had been subjected to electrophoresis and fluorography.

FIG. 3B illustrates the quantitation of eIF5A protein levels from islets from injected mice (control, si-Control and si-eIF5A) wherein the data represents the mean±SEM of 3 independent siRNA injections.

FIG. 3C illustrates the GSIS (glucose-stimulated insulin secretion) data of the islets from injected mice at the indicated glucose concentrations.

FIG. 3D illustrates the GSCa (glucose-stimulated Ca²⁺ mobilization) data of the islets from injected mice at the indicated glucose concentrations.

FIG. 3E illustrates the data of the islets from mice treated with a cocktail of cytokines (IL-1β, TNFα, IFNγ) for 4 hours and subjected to GSIS (glucose-stimulated insulin secretion) at the indicated glucose concentrations.

FIG. 3D illustrates the data of the islets from mice treated with a cocktail of cytokines (IL-1β, TNFα, IFNγ) for 4 hours and subjected to GSCa (glucose-stimulated Ca²⁺ mobilization) at the indicated glucose concentrations.

FIG. 4A illustrates data derived from islets of injected (vehicle or siRNAs) male C57BL/6J mice where the data is normalized to Actb mRNA levels, and reported as expression relative to vehicle injection.

FIG. 4B illustrates data derived from islets of injected (vehicle or siRNAs) male C57BL/6J mice after exposure to cytokines for 4 hours.

FIG. 4C illustrates data derived from islets from injected mice, untreated and treated with cytokines for 4 hours and then subjected to real-time RT-PCR for Nos2 mRNA.

FIG. 4D illustrates representative iNOS and actin immunoblots of islet extracts from injected mice treated with cytokines for 4 hours.

FIG. 5A illustrates a representative immunoblot of a mouse islet extract after being treated with GC7 overnight, pulsed with ³H-spermidine for 4 hours, and then subjected to electrophoresis and fluorography.

FIG. 5B illustrates a representative immunoblot of actin and eIF5A from INS-1 cell extract following overnight treatment with GC7, where the “*” identifies an upper band of decreasing intensity.

FIG. 5C illustrates a representative immunoblot of iNOS and actin from INS-1 cell extract.

FIG. 5D illustrates nitrite levels in INS-1 cell medium.

FIG. 5E illustrates Nos2 transcript levels in INS-1 cells.

FIG. 5F illustrates representative immunoblots of iNOS and actin from human islets.

FIG. 5G illustrates Nos2 transcript levels in human islets normalized to Actb mRNA levels and reported as fold-induction relative to non-cytokine, non-GC7-treatment.

FIG. 5H illustrates representative immunoblots of INS-1 cells treated with vehicle (untransfected) or transfected with the siRNAs indicated.

FIG. 6A illustrates GSCa and GSIS studies of INS-1 β cell function for cells not exposed to cytokines.

FIG. 6B illustrates GSCa and GSIS studies showing that the inhibition of hypusination preserves INS-1 β cell function following a 4 hour exposure to cytokines.

FIG. 6C illustrates GSCa and GSIS studies showing that the inhibition of hypusination preserves INS-1 β cell function following a combination of 125 μM GC7 and 4 hour exposure to cytokines.

FIG. 7A illustrates immunoblots for extracts from INS-1 β cells that had been transfected with GFP-eIF5A or GFP-eIF5A (K50A)mutant, where the extracts were immunoprecipitated (IP) with the indicated antibodies, prior to being immunoblotted for GFP, exportin 1/CRM1, and eIF5A.

FIG. 7B illustrates RT-PCR data for Nos2, Actb, Nfkb1, and Gapdh from RNA fractions (from INS-1 cells) that had been exposed to 4 hours of cytokine treatment after (a) being subjected to no pretreatment; (b) being treated 3 hours with leptomycin B (Lep B); and (c) being treated overnight with GC7.

FIG. 8A provides images (cells fixed and stained for eIF5A and visualized by fluorescence microscopy at 488 nm) and data (cytoplasmic/nuclear ratios of eIF5A) for INS-1 β cells after the cells were (1) (a) exposed to vehicle (untreated); (b) exposed to GC7 overnight; or (c) exposed to leptomycin B (LepB) for 3 hours; and (2) exposed to cytokine treatment for 4 hours or not exposed to cytokine treatment.

FIG. 8B provides images (cells fixed and stained for eIF5A and visualized by fluorescence microscopy at 488 nm) and data (cytoplasmic/nuclear ratios of eIF5A) for INS-1 β cells (1) after the cells were transfected with expression vectors encoding GFP fusions of either eIF5A or eIF5A (K50A) mutant and (2) exposed to cytokine treatment for 4 hours or not exposed to cytokine treatment.

FIG. 9A provides quantitative RT-PCR data on RNA isolated from INS-cells following (a) exposure to vehicle or GC7 overnight and (b) exposure to 4 hour vehicle or cytokine treatment prior to isolation of total RNA.

FIG. 9B provides quantitative RT-PCR data for immunoprecipitated RNA from INS-1 cells that had been (a) exposed to vehicle or GC7 overnight: (b) exposed to a 4 hour cytokine treatment; and (c) harvested for immunoprecipitation assays using either the eIF5A antibody or an isotype-matched control antibody (FLAG-M2).

FIG. 10A provides data from a glucose tolerance test on C57BL/6J mice subjected to (a) daily intraperitoneal injections of GC7 or control saline (for 7 days); and (b) 5 consecutive injections of low dose streptozotocin via intraperitoneal injection on day 7.

FIG. 10B provides data from a glucose tolerance test on C57BL/6J mice subjected to (a) daily delivery of GC7 or control saline through an implanted osmotic pump (for 7 days); and (b) 5 consecutive deliveries of low dose streptozotocin via an implanted osmotic pump on day 7.

FIG. 10C provides blood insulin levels determined during the GTT illustrated in FIG. 10A for each group of mice.

FIG. 10D provides β cell mass levels in mice utilized in the test described in FIG. 10A for each group of mice.

FIG. 10E provides stained pancreata cells from representative animals from each group described in FIG. 10A.

FIG. 10F provides serum levels of the indicated cytokines (IL-6, IL-13 and Rantes) described in FIG. 10A following intraperitoneal injection of the STZ-treated C57BL/6J mice with IL-1Ra.

FIG. 11A provides the results from a GTT administered on male NOD/Scid-IL-2Ry-null) mice that were either untreated or were administered a single dose of LPS concurrently with either saline or GC7.

FIG. 11B provides images of stained pancreata from representative animals from each group associated with FIG. 11A.

FIG. 12 illustrates a current model for eIF5A control of Nos2 translation illustrating a likely role of eIF5A in controlling Nos2 translation.

FIG. 13A provides quantitative RT-PCR data for the expression of the mRNAs encoding eIF5A1 and eIF5A2 in primary mouse and human islets.

FIG. 13B provides immunoblot analysis for the expression of actin, eIF5A2 and eIF5A1 proteins in extracts from the indicated cell types.

FIG. 14A provides electrophoresis and fluorography data for 3H-eIF5A-Hyp from INS-cells (a) treated with GC7 and 1 mM aminoguanidine; and (b) being pulsed with ³H-spermidine for 4 hours (upper panel) and representative immunoblots of actin and eIF5A from INS-1 cell extract following overnight following treatment with GC7 and 1 mM aminoguanidine.

FIG. 14B provides immunoblot analysis of iNOS and actin from INS-1 cells treated with GC7 and 1 mM aminoguanidine (upper panel) and quantitation of iNOS protein levels corrected for actin levels.

FIG. 14C illustrates the inability of low levels of GC7 to inhibit iNOS activity compared to aminoguanidine.

FIG. 14D provides cell viability data in the form of quantitative 2 color fluorescence analysis of live (calcein-AM-positive) and dead (ethidium homodimer-1-positive) cells (performed using fluorescence cytometry) illustrating that the percentage of dead INS-1 cells (˜4-5%) was unaffected by increasing GC7 concentrations between 0 to 125 μM after overnight exposure.

FIG. 14E provides the results of cell cycle analysis following fluorescence cytometry of INS-1 cells.

FIG. 15A provides immunoblot analysis for actin and eIF5A carried out on extracts from HeLa and INS-1 cells after the cells were pulsed with ³H-spermidine (4 hours), followed by periods of chase with 1 mM unlabeled spermidine, and after the extracts were subjected to electrophoresis on a 12% SDS-polyacrylamide gel and fluorography.

FIG. 15B provides a line graph illustrating quantitation of 3H-eIF5A-Hyp levels in the pulse chase experiments related to the experiments described for FIG. 15A.

FIG. 15C provides data from the electrophoresis and fluorography extracts from mouse and human islets which were similarly pulsed with ³H-spermidine for the times indicated.

FIG. 16 provides images of islets from pancreata of male C57BL/6J mice fixed, paraffin imbedded, and immunostained by TUNEL for dead cells following (1) treatment including the administration of (a) a control saline (untreated); (b) a low dose of STZ; or (c) GC7 and STZ solutions.

### DESCRIPTION

In both type 1 and type 2 diabetes, a key feature of islet dysfunction emanates from inflammatory cascades triggered by cytokine signaling. The subsequent production of iNOS and the generation of nitric oxide, among other mediators, causes defects in insulin release (7). This disclosure, identifies eIF5A-Hyp as a proximal regulator of iNOS production, and shows that eIF5A depletion as well as the inhibition of hypusination preserves islet glucose responsiveness in the presence of cytokine-induced stress.

eIF5A, previously known as eIF4D and IF-M2Ba, is a highly conserved 17 kDa protein that was originally characterized as a translation initiation factor promoting the formation of the first peptide bond in mRNA translation in vitro (50 and 51). However, its role as a general translational factor has seen diminishing enthusiasm over the years, as studies using yeast mutants have shown that eIF5A is not essential for general protein translation, but instead probably necessary for the translation of specific transcripts (52). More recently, eIF5A has been thought to function in the translation of mRNAs that encode proteins essential for the G1-S transition of the cell cycle (53), for cytotoxic stress responses (54), and for the propagation of human immunodeficiency virus (55). Thus, eIF5A is best positioned as a factor that controls the balance between cellular proliferation and death, depending upon the nature of cellular stress.

Whereas eIF5A is expressed in dendritic cells and is necessary for the nuclear-to-cytoplasmic transport of the mRNA encoding the maturation marker CD83, to date no role for eIF5A within the islet has been proposed. The present work shows that depletion of eIF5A (and its active hypusinated form) in islets using a previously characterized and specific siRNA (21) results in relatively preserved islet glucose responsiveness upon exposure to cytokines, as assessed by GSCa and GSIS. The phenotype, as observed, following a ˜50% decrease in the protein suggests that a nearly full complement of eIF5A-Hyp is necessary for the normal stress response to cytokines. In order to identify the initial pathways leading to islet dysfunction, brief incubation time with cytokines (4 h) were utilized, as more prolonged incubations may lead to convergence of multiple signaling effects resulting in eventual islet death. Whereas the gene encoding iNOS (Nos2) was upregulated in these islets, iNOS protein was strikingly suppressed in si-eIF5A-treated islets compared to controls. These results are consistent with prior reports that Nos2 transcription and translation can be independently regulated processes (56). The current data show for the first time that eIF5A is a factor central to Nos2 translation. eIF5A is the only protein known to contain the unique amino acid hypusine (57). Hypusine is formed post-translationally in a reaction catalyzed by DHS and DOHH and involving transfer of a 4-aminobutyl moiety from spermidine to Lys50 of eIF5A (15). The uniqueness of this modification in mammalian cells is reflected in the observation that only a single protein (eIF5A1/2) is detectable upon incubation of cells with ³H-spermidine (25). eIF5A and its hypusinated form exhibit prolonged half-lives (≧24 h) in many mammalian cells (39 and 40); strikingly, however, pulse-chase studies revealed that eIF5A-Hyp exhibits only a ˜6 h half-life in primary islets and islet-derived cell lines (this study). Interestingly, in some cell types the half-life of eIF5A acutely diminishes to as little as 30 min. in response to stressors such as heat shock (58 and 59), suggesting that both the cell type and environmental conditions can significantly influence the stability of the protein. In the present study, therefore, the islet β cell represents a unique case study for eIF5A-Hyp biology.

Upon incubation with an inhibitor to DOHH (mimosine) or DHS (GC7), both islets and INS-1 (832/13) β cells exhibit a cytokine-resistant phenotype virtually identical to knockdown of eIF5A protein by siRNA. Whereas islets exhibited a synchronized, sigmoidal pattern of Ca²⁺ accumulation in response to glucose, INS-1 cells demonstrated an asynchronous spiking pattern (38) that was effectively abolished upon incubation with cytokines but preserved upon co-incubation with GC7. Similar to findings in si-eIF5A-treated islets, incubation with GC7 resulted in a dose-dependent inhibition of iNOS protein levels in INS-1 cells and human islets. Taken together, the current studies with DHS inhibition not only support the specificity of the findings using si-eIF5A in islets, but also emphasize the importance of hypusination in the action of eIF5A. Hypusination appears to facilitate some protein-protein interactions and RNA binding by eIF5A (45 and 47). With regard to the latter, a potential eIF5A binding sequence within the Nos2 mRNA was identified and evidence provided that only eIF5A-Hyp physically associates with Nos2 mRNA, but significantly less so or not at all with mRNAs for other NFkB targeted genes. The findings therefore identify Nos2 mRNA as a novel and specific target for eIF5A action.

To determine how RNA binding might facilitate Nos2 translation, the possibility was considered that eIF5A aids in the nuclear to cytoplasmic transport of Nos2 mRNA. Nucleo-cytoplasmic shuttling of eIF5A has been observed by several groups, and the suggestion made that the hypusinated form may be compartmentalized differently from the unhypusinated form (46). Other studies suggest that eIF5A interacts with the nuclear export receptor exportin1/CRM1 and is required for HIV Rev protein-mediated viral RNA export and for the export of CD83 mRNA in dendritic cells (24, 44, 60, and 61). Exportin1/CRM1 serves as a cell context-dependent transporter for certain mRNAs (62), but notably it mediates (in part) the nucleo-cytoplasmic transport of Nos2 (63). The present work has shown that eIF5A forms a complex with exportin1/CRM1 in a manner that is not hypusine dependent, but that Nos2 mRNA nuclear export is at least partially dependent upon both exportin1/CRM1 and hypusination.

It is recognized, however, that the nucleo-cytoplasmic shuttling of eIF5A has been challenged by other groups (42, and 43), and still others purport an interaction between eIF5A and exportin4 (45). The possibility cannot be excluded, however, that exportin4 may also play a role in the transport of Nos2, considering that leptomycin B inhibition of exportin1/CRM1 blocked only ˜50% of Nos2 export in the present study. Nuclear-cytoplasmic shuttling may not be the only mechanism, by which eIF5A-Hyp controls Nos2 translation; although inhibition of hypusination blocked about 50% of Nos2 nuclear export, >90% of iNOS protein production was reduced. This finding is consistent with eIF5A-Hyp being required for linking Nos2 mRNA to the translational machinery. Prior studies have shown that yeast homolog of eIF5A interacts directly with the components of the translational machinery and is necessary for translational elongation (51 and 64).

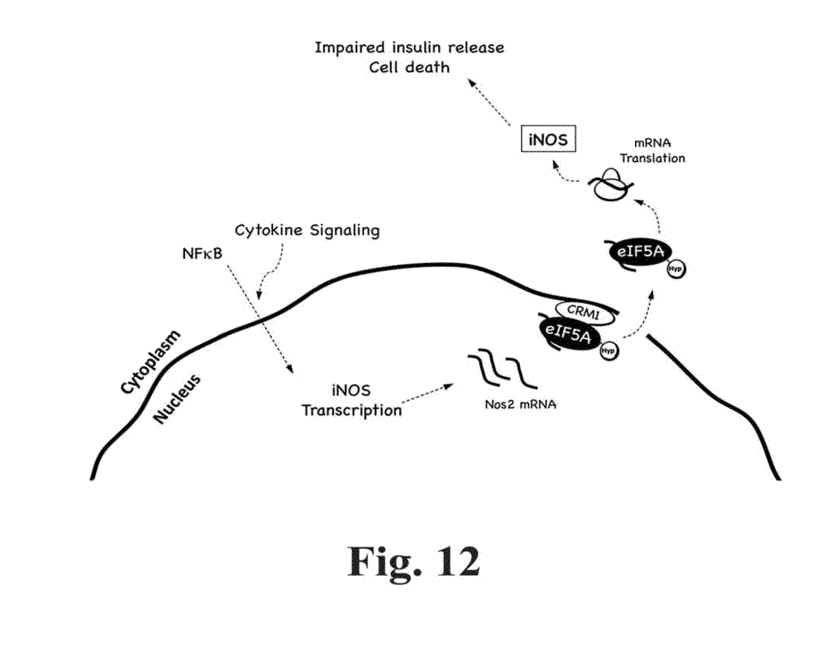

The physiologic relevance of these findings are supported by the studies in vivo, which demonstrated that si-eIF5A injected mice and GC7-treated mice were more resistant to STZ-induced islet dysfunction and hyperglycemia than controls. The studies in vivo closely parallel the results of Nos2-null mice, which also showed resistance to STZ and relative islet preservation (30). Although the mechanism of low dose STZ-induced islet dysfunction and hyperglycemia is complex, studies point to a toxic effect of STZ on islets, which causes the influx of inflammatory cells with local release of cytokines (27 and 28). This mechanism (thought to be similar to that seen in type 1 diabetes) is supported by current findings that the hyperglycemic effect of STZ in immunocompetent mice can be mitigated by the IL-1Ra anakinra Thus, the current findings on the effect of eIF5A in STZ diabetes is similar to those observed upon knockout of other pro-inflammatory factors residing in the Idd4 locus, such as 12-lipoxygenase and iNOS (30, 65, and 66). Finally, the protective effect of DHS inhibition is borne out in the present LPS injection studies in immunodeficient NOD/Scid-(IL-2Rγ-null) mice. These studies support a primary role for DHS and eIF5A-Hyp in the inflammatory response within the islet (rather than a secondary effect upon immune cells). Taken together, the present data identify a novel role for eIF5A and its hypusinating enzyme DHS in effecting the early islet response to cytokine-induced stress. The present study supports a model (FIG. 12) whereby cytokine stimulation collectively leads to rapid induction of Nos2 gene transcription via activation of the transcription factor NFkB. This, in turn, leads to the generation of Nos2 mRNA, which is transported across the nuclear membrane in an exportin1/CRM1-eIF5A-dependent fashion. Ongoing binding to eIF5A in the cytoplasm may ensure transcript delivery to ribosomes, where translation is facilitated. A key component in this model is the hypusination of eIF5A, which is necessary for the binding to Nos2 transcripts and for the translocation of the complex across the nuclear membrane. Importantly, it is recognized that this model is not likely exhaustive with respect to the phenotypes in vivo observed in this study. For example, eIF5A-Hyp has recently been shown to mediate translational elongation in yeast (51); as such, it is possible that the pro-inflammatory effects of eIF5A-Hyp may be related to regulation of as yet other unidentified transcripts. The present studies therefore suggest that targeting of DHS represents a novel therapeutic strategy to protect pancreatic islets from inflammation.

eIF5A1, but not eIF5A2, is Expressed in Pancreatic Islets

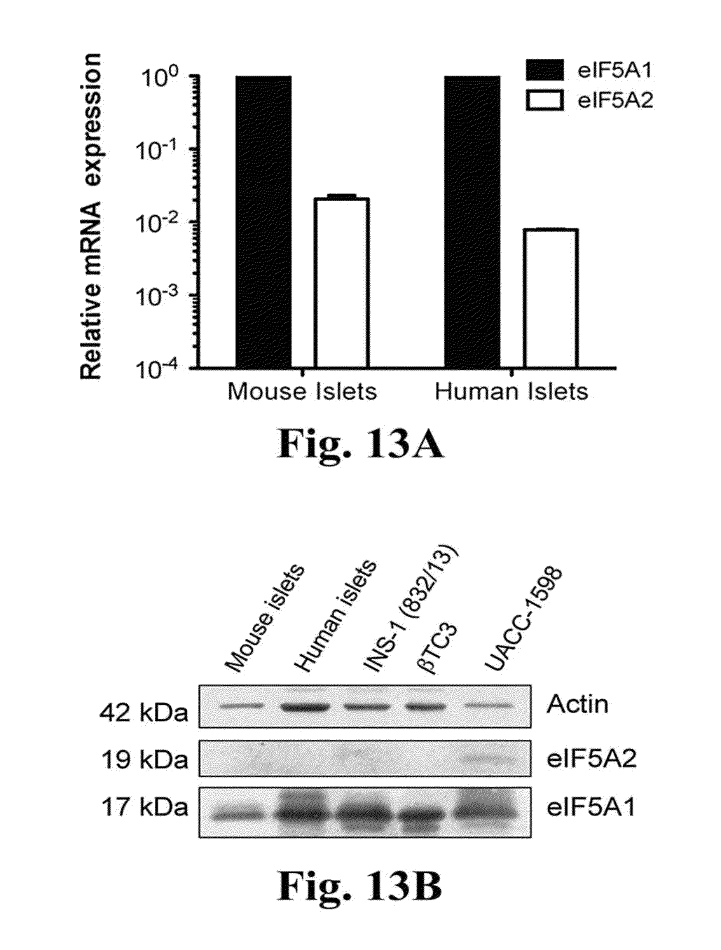

eIF5A exists as two isoforms, eIF5A1 and eIF5A2, which exhibit differing tissue distributions (25 and 26). Quantitative real-time RT-PCR and immunoblots were performed to determine whether one or both isoforms are present in islets and islet-derived cell lines (FIG. 13B). The mRNA encoding for eIF5A2 is expressed at approximately 50-100-fold lower levels than that encoding eIF5A1 in mouse and human primary islets (FIG. 13A). Immunoblots were next performed using antibodies against each isoform of eIF5A and protein extracts from islets and islet cell-derived cell lines (FIG. 13B); whereas the control ovarian cancer-derived cell line UACC-1598 contains both eIF5A1 and eIF5A2 (see also ref. (25), islets and islet-derived cell lines show detectable protein expression of only eIF5A1. The results indicate that eIF5A1 is the major isoform expressed in pancreatic islets (and this isoform will henceforth simply be referred to as “eIF5A”).

Depletion of eIF5A Protects Mice Against Multiple Low-Dose Streptozotocin (STZ)-Induced Hyperglycemia and Islet Loss

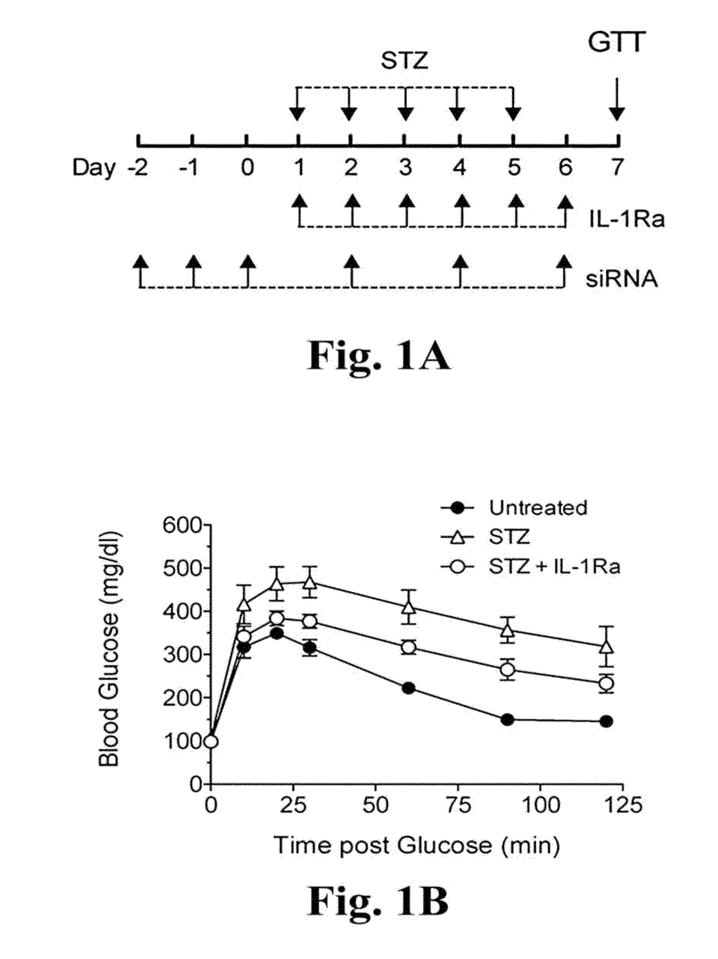

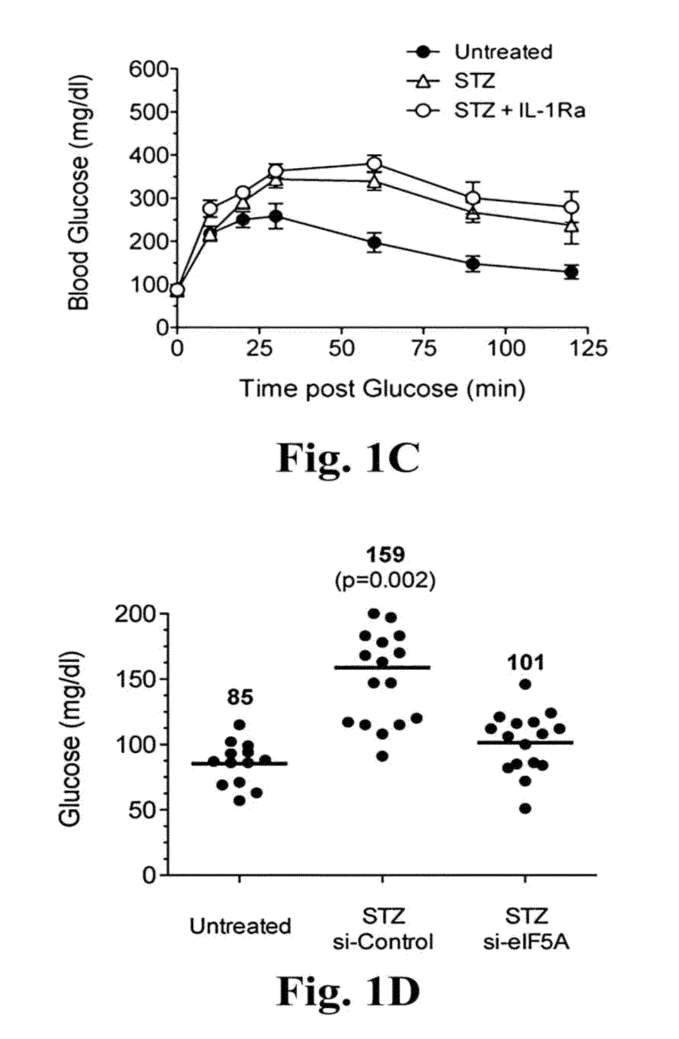

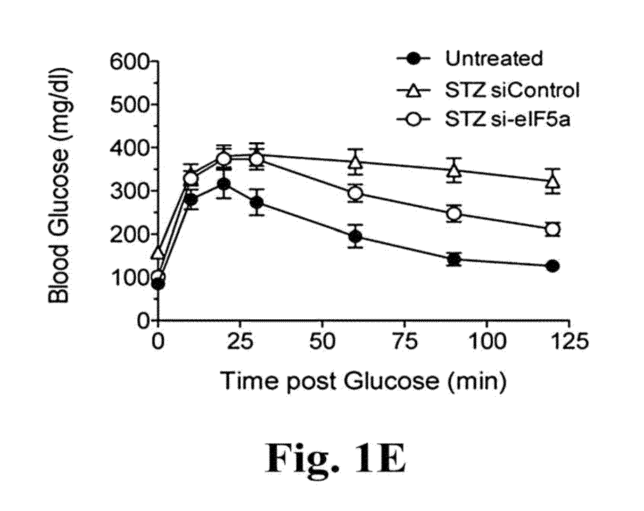

To test the role of eIF5A in cytokine-mediated islet dysfunction, efforts were made to identify a mouse model of islet inflammation. The multiple low dose streptozotocin (STZ) model, in which mice are subjected to five daily intraperitoneal doses (at 55 mg/kg body weight) of STZ, is considered to provoke local islet inflammation and cytokine release, in part through the recruitment of CD11c+ dendritic cells that release pro-inflammatory cytokines in the area of STZ-induced cellular destruction (27 and 28). The dependence of this model on cytokine release from immune cells was demonstrated by subjecting both C57BL/6Jmice (with intact immune system) and NOD/Scid-(IL-2Rγ-null) mice (without innate or adaptive immune systems) to multiple low-dose STZ as shown in the schematic in FIG. 1A. FIGS. 1B and 1C show that both immune-competent (FIG. 1B) and -incompetent (FIG. 1C) mice exhibit impaired intraperitoneal glucose tolerance tests (IPGTTs) following STZ injections; however, concurrent treatment of mice with IL-1Ra attenuated glucose intolerance only in immune-competent mice. These data indicate that multiple low-dose STZ exhibits at least two components contributing to islet dysfunction: one component that is dependent upon cytokine release from immune cells (as observed in C57BL/6J mice) and a second component that involves the known direct toxic, DNA-alkylating effect of STZ (29) (as observed in both C57BL/6J mice and NOD/Scid-(IL-2Rγ-null) mice).

Clarification of the role for eIF5A during the pathogenesis of islet inflammation in diabetes involved depleting mice of eIF5A using the in vivo RNA interference approach of Moore et al. (21).

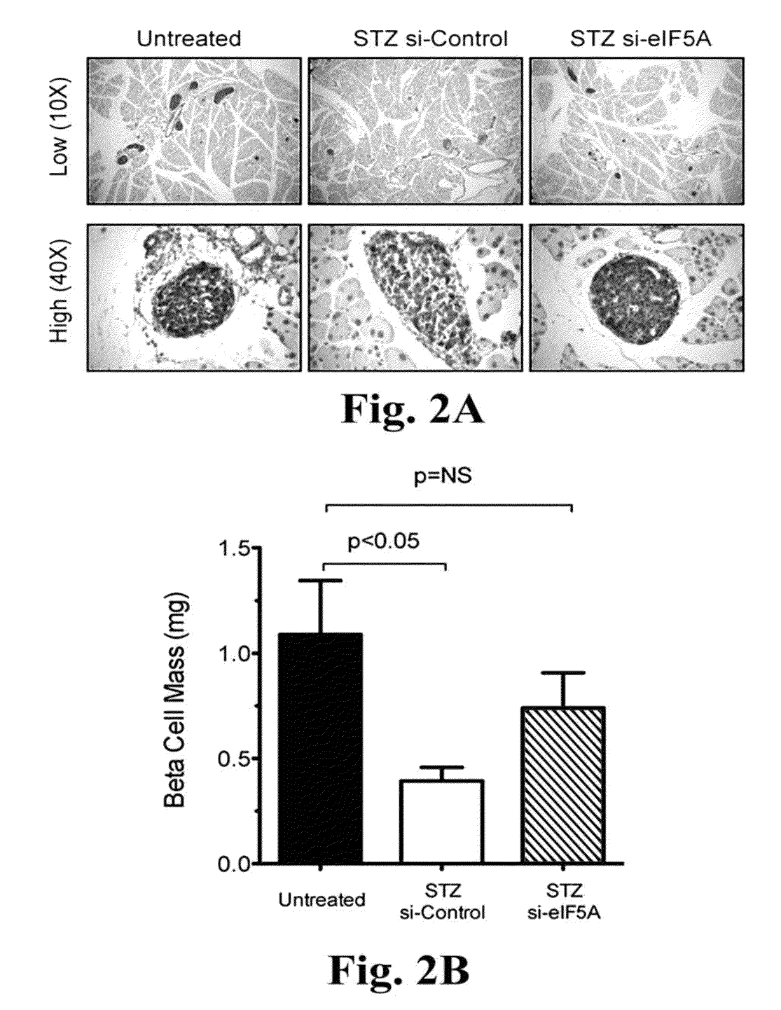

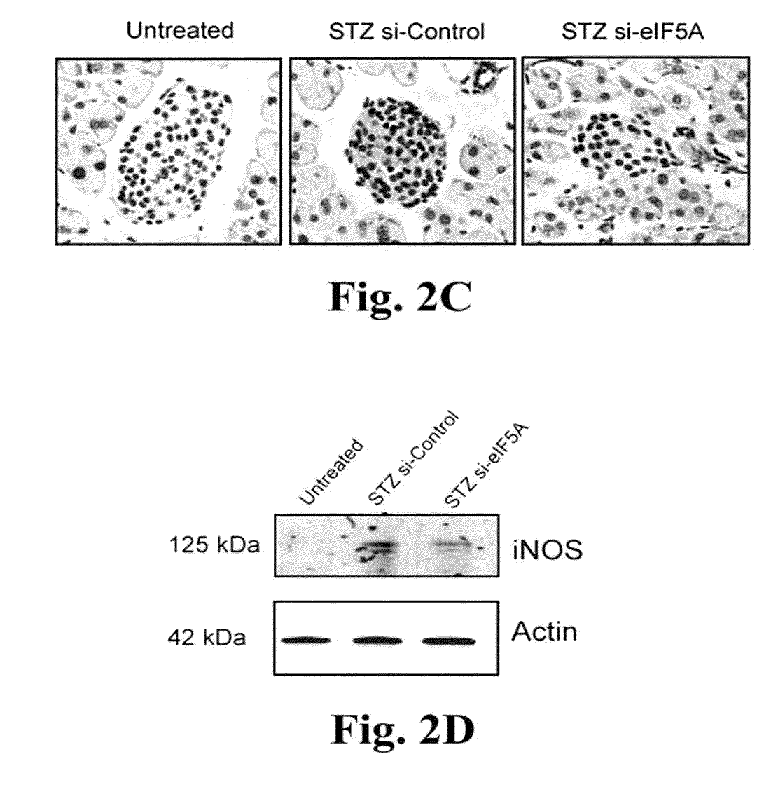

C57BL/6J mice were injected intraperitoneally with either stabilized siRNA against eIF5A (si-eIF5A) or control siRNA (si-Control) daily for three days, then subjected to the multiple low-dose STZ protocol (see schematic in FIG. 1a). As illustrated in FIG. 1D, mice treated with STZ exhibited higher fasting blood glucose levels following STZ injection compared to untreated mice, the average fasting blood glucoses of mice injected with si-eIF5A (101 mg/dl) was significantly lower than that of mice injected with si-Control (159 mg/dl). Consistent with these fasting blood glucose data, IPGTTs demonstrated that si-eIF5A-treated mice exhibited improved glucose tolerance compared to si-Control-injected mice (FIG. 1E). The effect of administering si-eIF5A on islet viability following STZ treatment was determined by performing immunohistochemical analysis of the pancreata of mice. As shown in FIG. 2A, si-Control-injected mice showed an apparent reduction in islet number and weaker insulin staining (i.e. degranulation) relative to mice not given STZ. In contrast, si-eIF5A-injected mice exhibited relatively preserved islet number and insulin granularity. To quantify the differences in islet mass between STZ-treated and untreated animals, morphometry of insulin-stained pancreatic sections was performed. The si-Control-injected mice exhibited 2.8-fold reduction of β cell mass compared to non-STZ-treated controls, whereas si-eIF5A-injected animals demonstrated only a statistically insignificant 1.4-fold reduction (FIG. 2B). These data support the view that eIF5A is required for the early events that lead to eventual islet dysfunction following STZ treatment.

eIF5A Mediates Cytokine Toxicity and iNOS Production in Islets

As noted, low-dose STZ-induced islet dysfunction involves at least two mechanisms: an inflammatory mechanism that is dependent upon cytokine release from immune cells and a direct toxic mechanism on the islet itself. To distinguish whether eIF5A depletion affected one or both of these mechanisms, injections of si-eIF5A were performed on NOD/Scid-(IL-2Rγ-null) mice, which lack fully functional immune cells and therefore react to STZ via a direct islet toxicity. Like IL-1Ra injections, si-eIF5A injections did not protect against glucose intolerance in NOD/Scid-(IL-2Rγ-null) mice (data not shown), consistent with eIF5A being involved in only the cytokine-mediated component of STZ-induced islet dysfunction. FIG. 2C shows that immunohistochemical staining for iNOS persisted in islets of si-Control-injected animals at the end of the study, but was undetectable in si-eIF5A-injected animals. Next, islets from 3 C57BL/6J mice per group 24 h after the initial STZ treatment were isolated and pooled and their iNOS content determined by immunoblot. As shown in FIG. 2D, iNOS was rapidly induced in islets within 24 h of the first STZ treatment in si-Control mice, but this induction was attenuated in si-eIF5A mice. These results indicate that cytokine-induced iNOS production underlies the early events of STZ-induced islet dysfunction and loss, and that si-eIF5A treatment mitigates iNOS production.

Depletion of eIF5A Protects Islets from Cytokine-Induced Dysfunction In Vitro

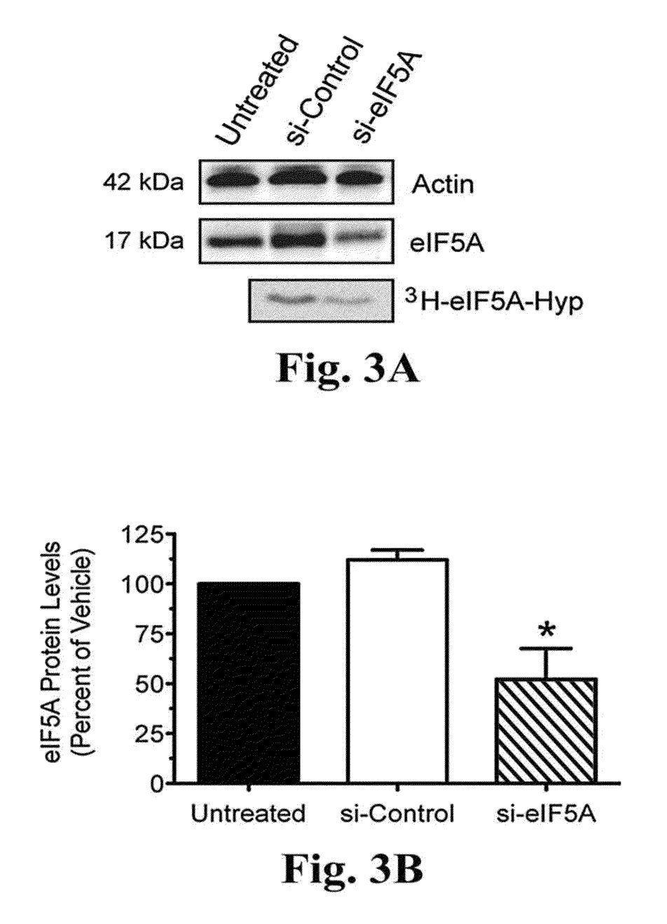

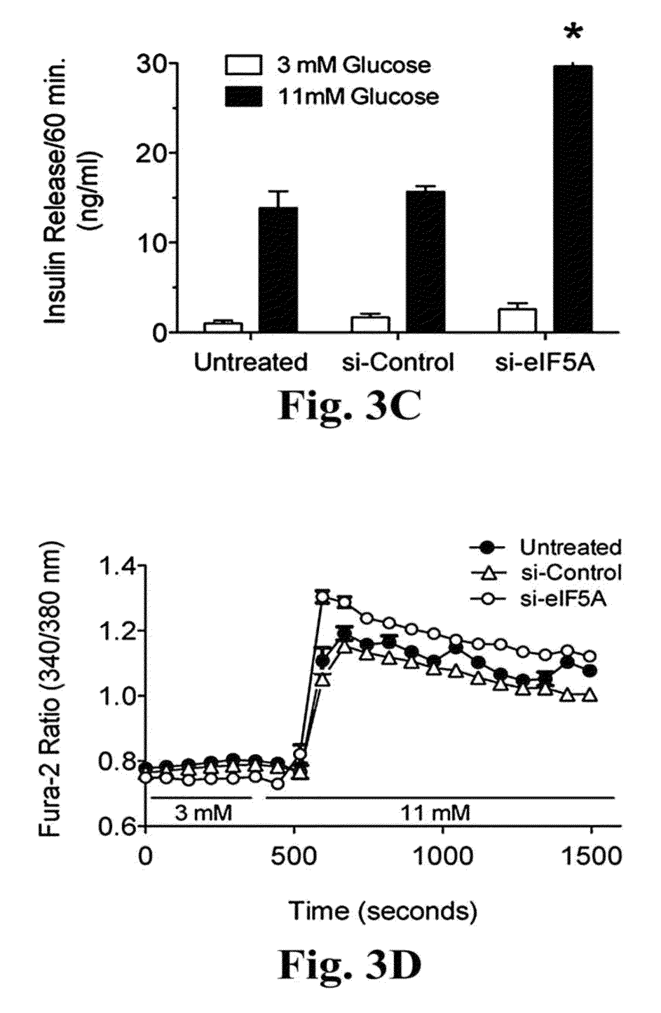

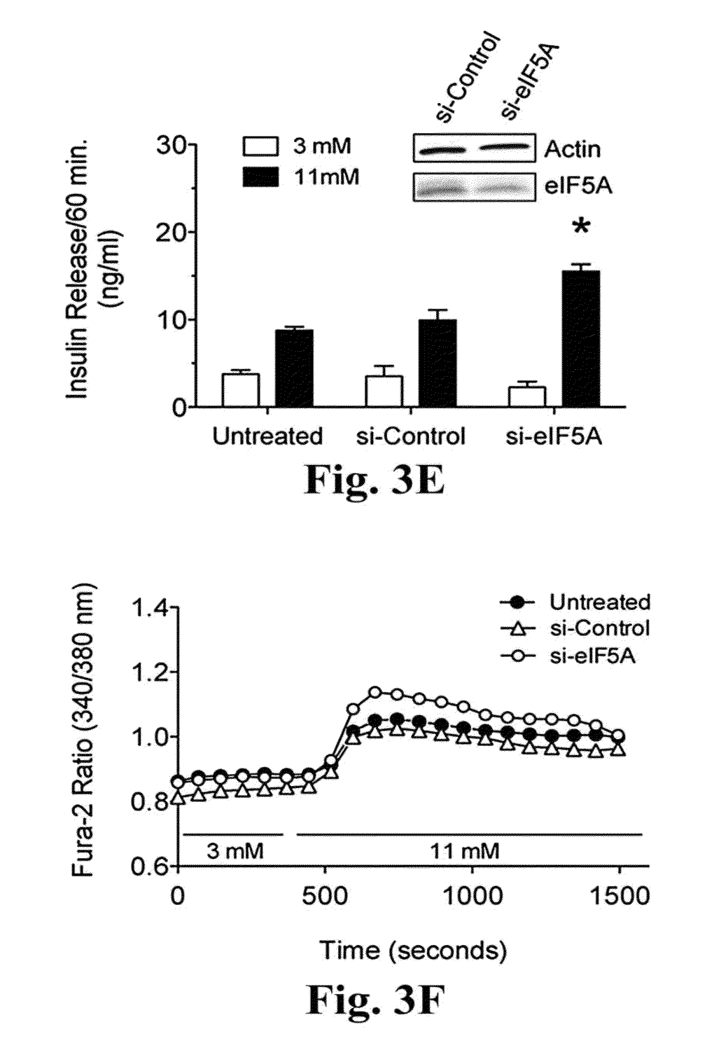

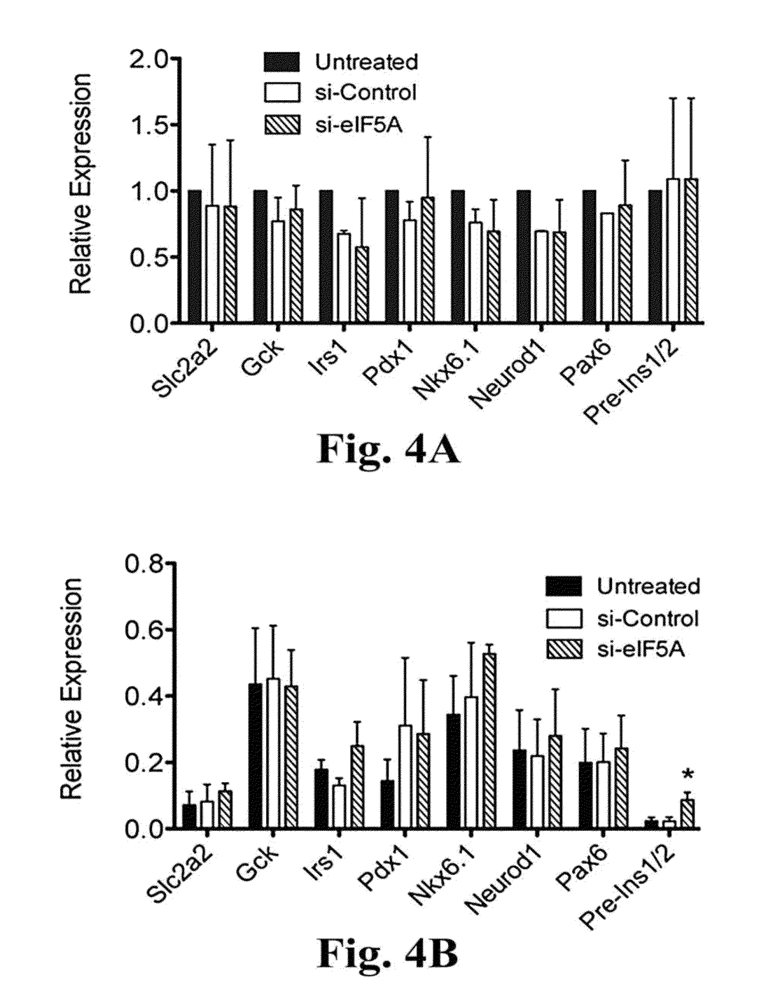

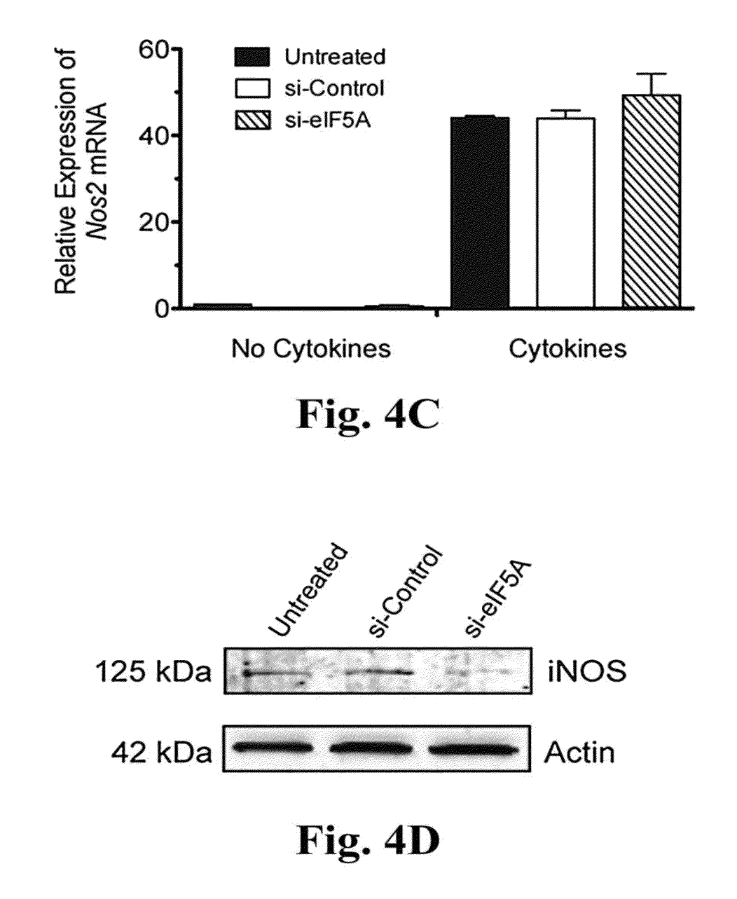

Our studies indicate a role for eIF5A in mediating systemic cytokine responses, but do not directly implicate its action in pancreatic islets. To clarify this, islets were isolated from siRNA-injected mice and their function assessed in vitro. As shown in FIGS. 3A and 3B, intraperitoneal injection of si-eIF5A for three days led to a ˜50% reduction of eIF5A in islets, as determined by immunoblot. ³H-spermidine incubations revealed a similar reduction in the active, hypusinated form of eIF5A (eIF5A-Hyp) in islets of si-eIF5A mice (FIG. 3A). This depletion corresponded to a significant improvement in islet response to glucose stimulation, as determined by glucose-stimulated insulin secretion (GSIS) studies (FIG. 3C). In order to further characterize this improvement in islet glucose responsiveness, glucose-stimulated Ca2+ mobilization (GSCa) studies were also performed. GSCa is a measure of islet glucose sensitivity that captures the dynamics of the islet glucose response, which is similar to GSIS (31). The GSCa, as measured by the change in fura-2 AM fluorescence ratio after glucose stimulation, was increased in si-eIF5A-treated islets compared to controls, reflecting the functional improvement seen in GSIS (FIG. 3D). This improved GSCa relative to controls persisted 48 and 72 hours post-isolation (data not shown).

The improvement in islet function following the knockdown of eIF5A indicates that eIF5A contributes to the stress of collagenase exposure during islet isolation. To directly assess whether eIF5A contributes to stress responses in the islet, islets were exposed to a cocktail of proinflammatory cytokines (IL-1β, TNFα, IFNγ)—a condition believed to mimic islet inflammation as seen in multiple low-dose STZ and the major forms of diabetes (32). A short cytokine exposure (4 h) was utilized to assess early impairment in islet function independent of islet cell death. Although islets from all three groups (vehicle, si-Control, or si-eIF5A) exhibited some impairment in GSIS and GSCa compared to their non-cytokine-exposed counterparts, islets from mice treated with si-eIF5A showed significant preservation of GSIS and GSCa compared to controls (see FIGS. 3E and 3F). As shown in the inset to FIG. 3E, eIF5A levels remained persistently lower in the si-eIF5A group even after cytokine exposure. Further, islet viability did not differ significantly between groups in these studies, as determined by ethidium homodimer-1/calcein-AM uptake studies, and no activation of caspase 3 was observed during this short time course (data not shown). These data indicate that eIF5A contributes to acute islet dysfunction in response to proinflammatory cytokines, even prior to overt cell death.

Endogenous eIF5A Promotes Translation of the mRNA Encoding iNOS in Primary Islets

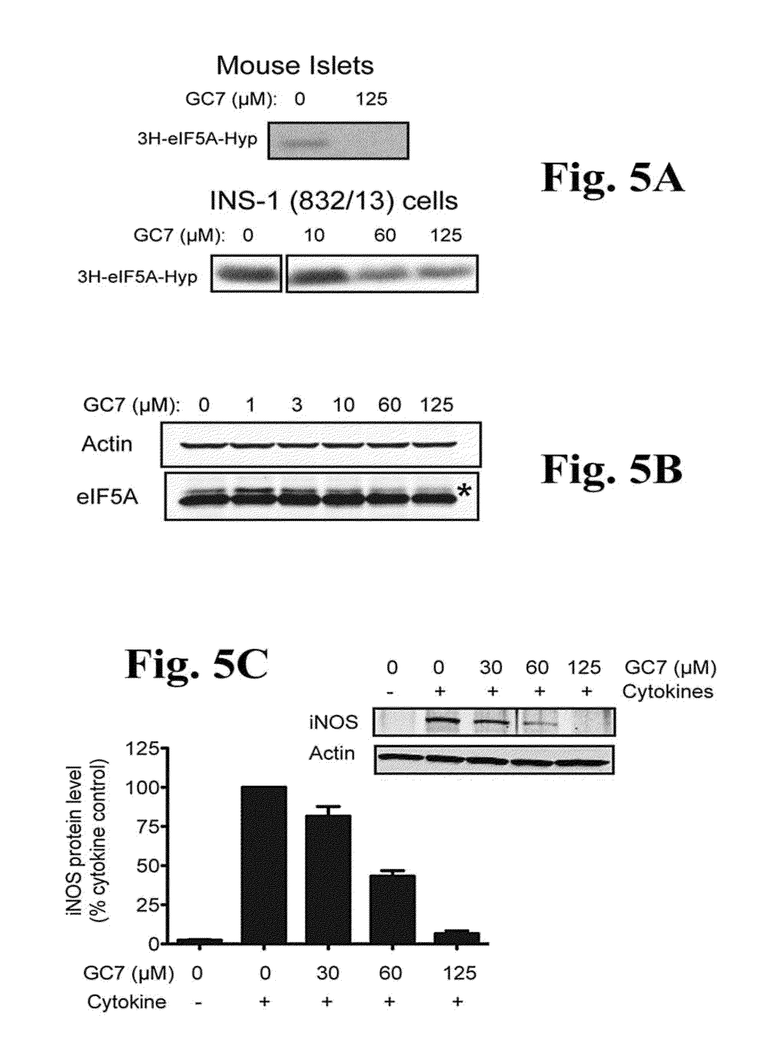

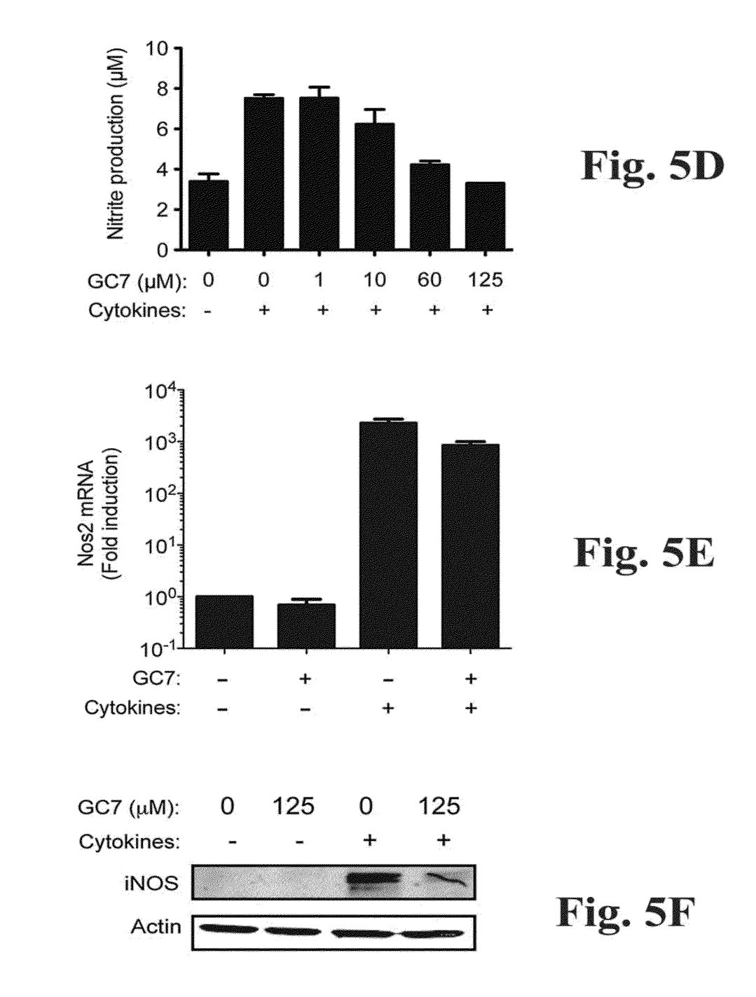

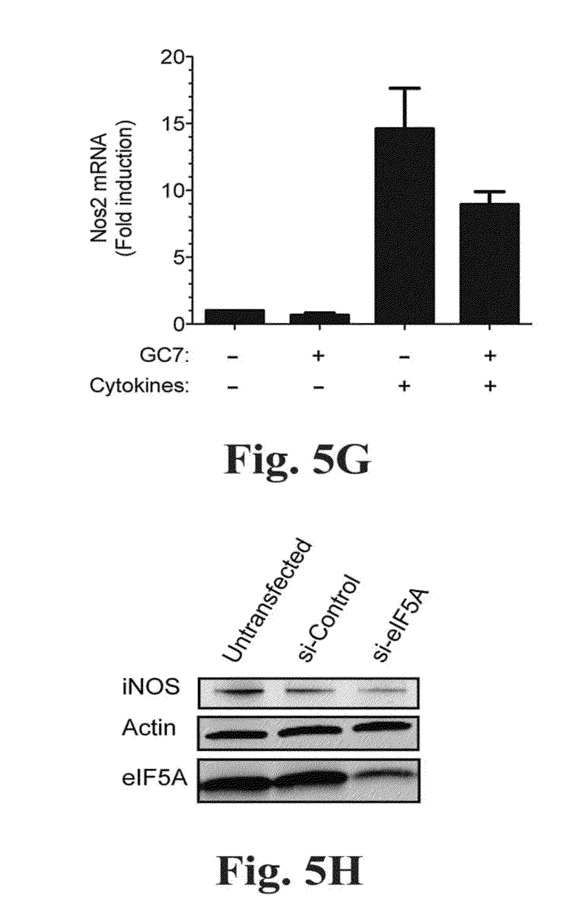

To better understand the protective effect of eIF5A knockdown in primary islets, the transcriptional response by real-time reverse transcriptase (RT)-PCR of genes known to mediate glucose responsiveness and β cell growth in islets exposed to vehicle vs. cytokines were evaluated (FIGS. 5A and 5B). Although there was relative preservation of Ins1/2 pre-mRNA in si-eIF5A-treated islets (consistent with the relative preservation of GSIS in these islets), there were otherwise no significant differences in the transcription of any of the known genes involved in glucose response (Slc2a2, Gck), insulin signaling (Irs1), or β cell growth/differentiation (Pdx1, Nkx6-1, NeuroD1, Pax6). Instead, 2-10-fold reductions in the steady-state levels of all of these genes occurred in response to cytokines in all siRNA treatment groups.

Because nitric oxide is known to be a primary effector of islet dysfunction in response to cytokine-induced stress, the transcription of the gene encoding iNOS (Nos2) was also examined. As shown in FIG. 5C, unlike the other genes examined above, a striking 40-fold activation of Nos2 mRNA in all siRNA treatment groups was observed in response to cytokines Notably, however, whereas iNOS protein was coordinately induced in islets of mice treated with vehicle and control siRNA, induction of iNOS protein was significantly attenuated in islets from si-eIF5A-treated mice (FIG. 5D). These data provide evidence that eIF5A may be a crucial regulator of Nos2 mRNA translation in primary islets.

Hypusination of eIF5A is Essential for Nos2 mRNA Translation.

eIF5A1 and eIF5A2 are the only proteins known to contain the unique amino acid hypusine at position 50. Hypusine is formed as a posttranslational modification of residue Lys50 in a reaction requiring spermidine and catalyzed by the sequential actions of the enzymes deoxyhypusine synthase (DHS) and deoxyhypusine hydroxylase (DOHH) (Chen, K Y, and Liu, A Y. Biochemistry and function of hypusine formation on eukaryotic initiation factor 5A. Biol Signals. 1997; 6(3):105-109). DHS is the rate limiting enzyme of this biosynthetic pathway, and small molecule inhibitors of this enzyme have been used to specifically inhibit the activity of eIF5A (24 and 35). To further investigate the mechanism of eIF5A-regulated translation of Nos2, studies were performed following overnight incubation of cells with mimosine (an inhibitor of DOHH) and GC7 (an inhibitor of DHS). Incubation with cytokines following increasing concentrations of either mimosine or GC7 resulted in a dose-dependent attenuation of iNOS protein. FIG. 5A shows that GC7 at 125 μM effectively inhibits new hypusine formation (as determined by ³H-spermidine incorporation) in mouse islets and in the rat cytokine-responsive β cell-derived line INS-1 (832/13) (10). Interestingly, GC7 incubation also depletes the steady-state level of an eIF5A species as observed by immunoblot (identified by an asterisk in FIG. 5B); this latter species may represent a form of eIF5A-Hyp akin to that seen in 2-D gels (36). Similar to data in primary islets, the immunoblot in FIG. 5C (lanes 1 and 2) demonstrates that a 4 h incubation of INS-1 cells with cytokines results in the induction of iNOS. However, incubation with cytokines following increasing concentrations of GC7 resulted in a dose-dependent attenuation of iNOS protein (FIG. 5C) as well as nitrite release into the medium (FIG. 5D), with the greatest inhibition observed at 125 μM GC7. As with the siRNA studies in islets, the block in iNOS production by GC7 appeared to be at the level of mRNA translation, as Nos2 transcript levels remained disproportionately elevated even in the presence of 125 μM GC7 (FIG. 5E). Similar data using GC7 were obtained in primary mouse islets (data not shown) and in human islets (FIGS. 5F and 5G). To verify that the effect of GC7 was due to inhibition of eIFSA action, siRNA knockdown of eIFSA in INS-1 cells (FIG. 5H) was performed demonstrating that cytokine-mediated iNOS induction was indeed attenuated.

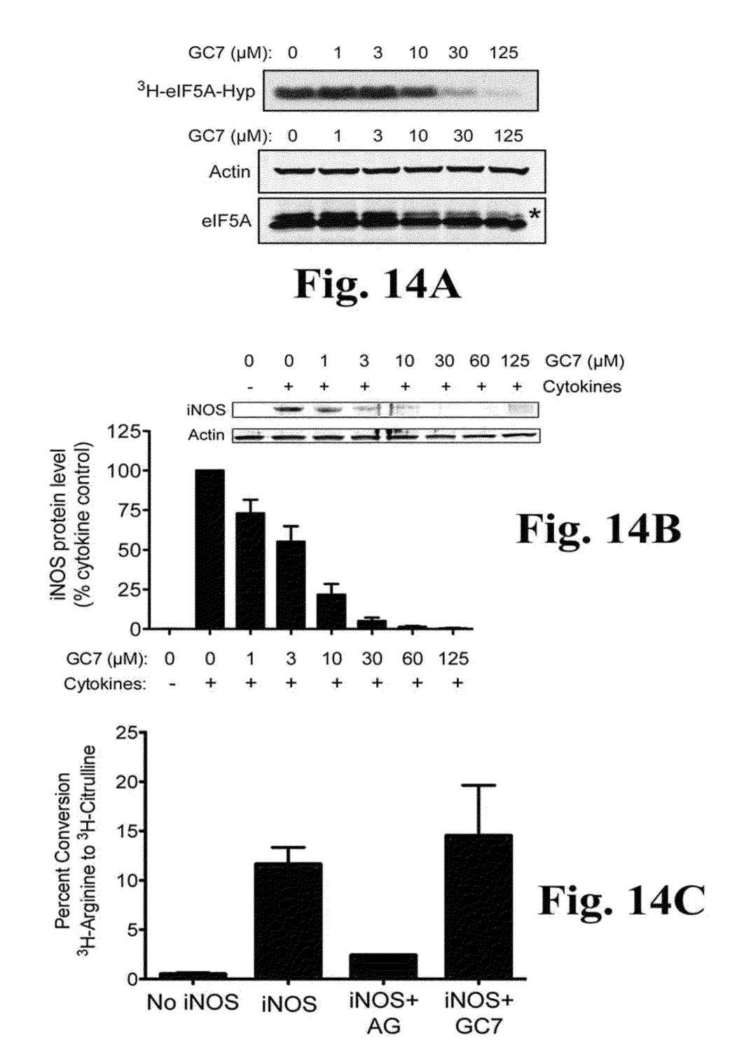

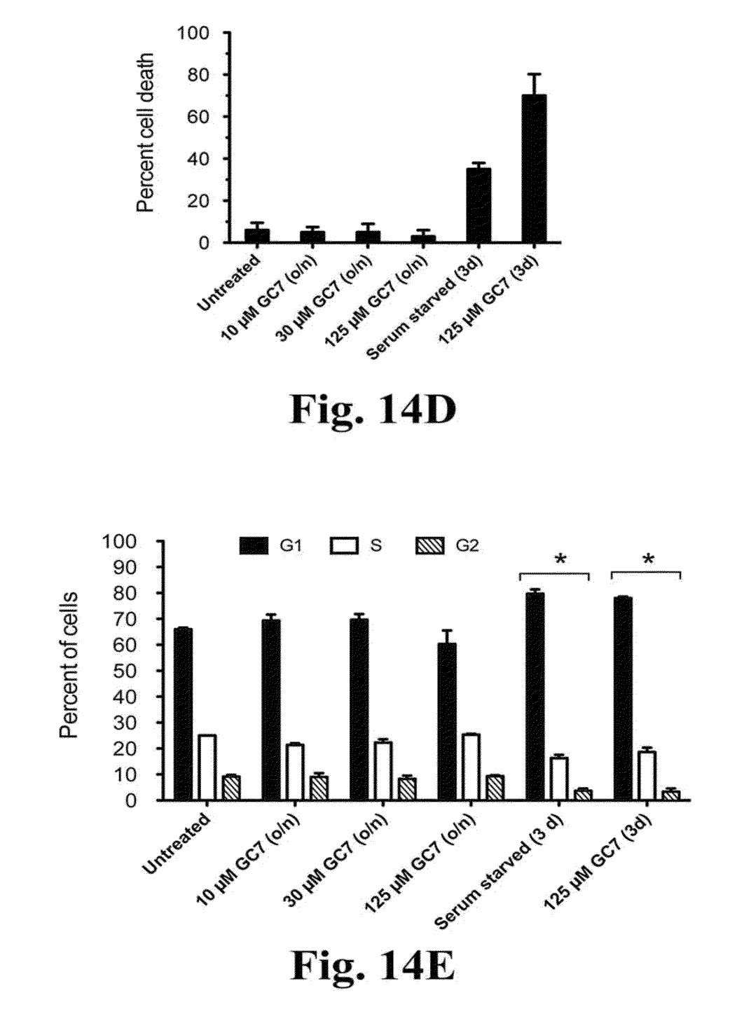

Notably, in the studies described above, concentrations of GC7 in excess of 30 μM were required to substantially block iNOS, raising questions about the specificity of the drug and possible effects of the drug on cell viability at these concentrations. Because GC7 is known to be inactivated by the action amine oxidases, which are abundant in serum, typical studies with GC7 in the literature have used aminoguanidine to inhibit amine oxidases (16). When co-incubated with 1 mM aminoguanidine, inhibition of new hypusine synthesis was observed at much lower concentrations (along with reductions in the steady-state level of the eIF5A species observed by immunoblot) (FIG. 14A). Under conditions of aminoguanidine co-incubation, inhibition of iNOS was observed with GC7 concentrations in the 3 μM range (FIG. 14B). Because aminoguanidine is also an effective inhibitor of iNOS catalytic activity (ref 37) and can therefore confound interpretation of our data, subsequent studies were carried out without the use of aminoguanidine and with higher concentrations of GC7 (125 μM) instead. At these concentrations, GC7 does not appear to inhibit iNOS activity (FIG. 14C). To rule out possible adverse effects of GC7 on cell viability, quantitative 2 color fluorescence analysis of live (calcein-AM-positive) and dead (ethidium homodimer-1-positive) cells were performed using fluorescence cytometry. From these studies it was found that the percentage of dead INS-1 cells (˜4-5%) was unaffected by increasing GC7 concentrations between 0 to 125 μM after overnight exposure (FIG. 14D); notably, however, 3 days exposure to 125 μM GC7 or serum starvation led to a dramatic increase in percentage of dead cells (70% and 30%, respectively) (FIG. 14D). Cell cycle analysis revealed that overnight GC7 treatment (at any concentration) had no significant effect on cell cycle progression (G1-S) in INS-1 cells; however, a cell cycle block was clearly apparent following 3 days serum depletion or 3 days GC7 exposure (FIG. 14E). These data are consistent with previous studies that implicate a role for eIF5A-Hyp in cell cycle progression in the long term (16), but also verify that our short-term overnight incubations with GC7 do not significantly affect INS-1 cell viability or cell cycle.

### Inhibition of Hypusination Protects Against Cytokine-Induced β Cell Dysfunction In Vitro.

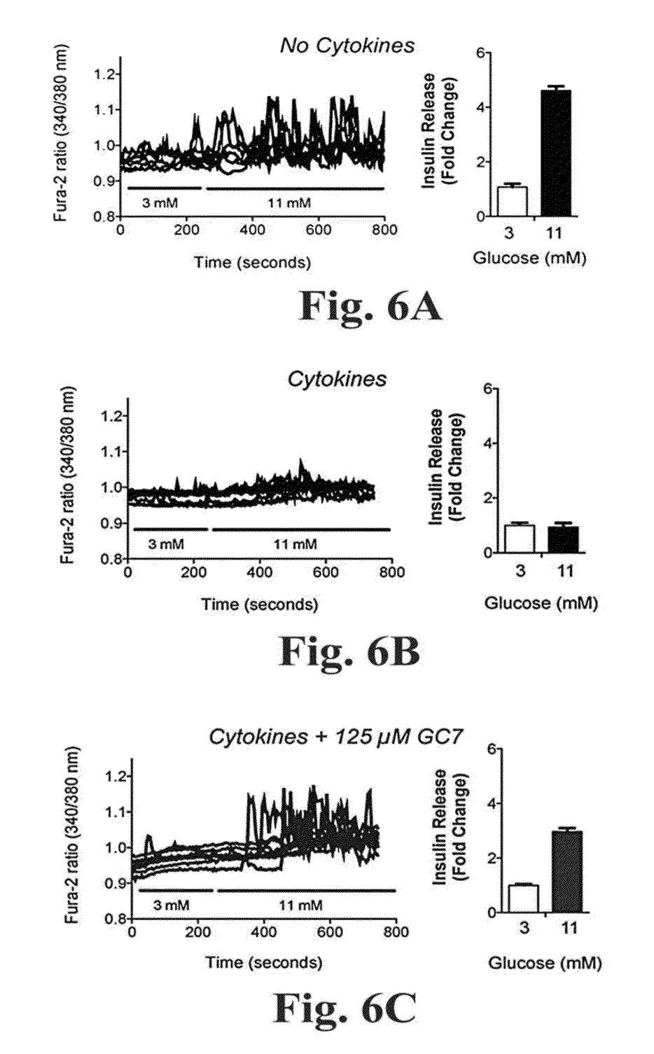

GSCa and GSIS studies were carried out to assess INS-1 cell function in the presence of cytokines and GC7. As shown in FIG. 6A (left panel), unlike intact islets, INS-1 cells exhibit an asynchronous GSCa pattern that is closely correlated with GSIS (FIG. 6A, right panel). This asynchronous pattern may be related to the dispersed nature of insulinoma cells compared to f3 cells in an intact islet. FIG. 6B shows that GSCa and GSIS are virtually abolished upon incubation with cytokines. Preincubation with 125 μM GC7, however, reverses the suppressive effect of cytokines on GSCa and GSIS almost completely (FIG. 6C). These results indicate that hypusination of eIF5A is crucial to promoting Nos2 translation and β cell dysfunction in response to acute exposure to the cytokines.

eIF5A-Hyp Exhibits a Relatively Short Half-Life in Islet β Cells

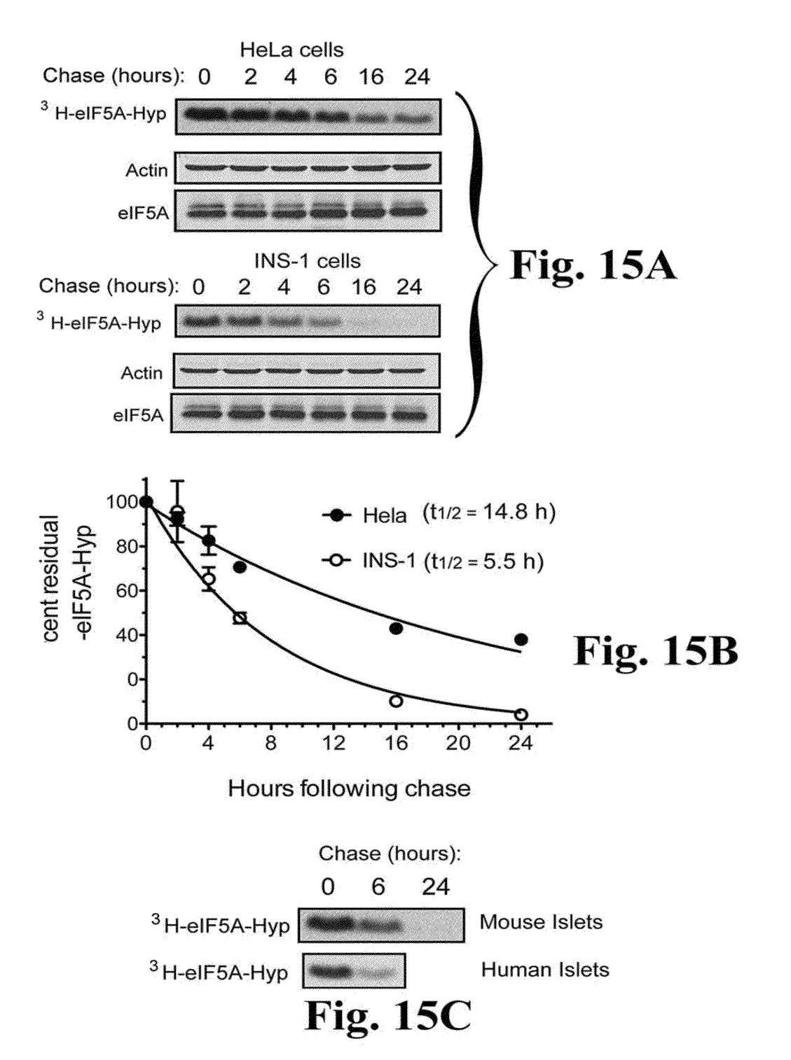

The abundance of data in the literature indicates that eIF5A and eIF5A-Hyp exhibit prolonged half-lives (>24 hours) in some mammalian cells (39 and 40). This prolonged half-life appears seemingly at odds with our relatively rapid inhibition of eIF5A action by overnight GC7 pretreatment, and suggests that either the half-life of eIF5A-Hyp is different in islet β cells and/or that de novo hypusination is required for the acute cytokine effects observed in our studies. To assess the half-life of eIF5A-Hyp in β cells and primary islets, pulse-chase experiments were performed in INS-1 cells and primary islets using ³H-spermidine, and tracked eIF5A-Hyp levels by fluorography following chase periods using polyacrylamide gel electrophoresis (36). As shown in FIGS. 15A and 15B, whereas in HeLa cells eIF5A-Hyp exhibits a ˜15 h half-life, the corresponding half-life in INS-1 β cells is only ˜6 h. In these experiments, no evidence of cellular death was observed by ethidium homodimer-1/calcein-AM uptake (data not shown). Pulse-chase experiments in both mouse and human islets reveal that eIF5A-Hyp is depleted by ˜50% at 6 h following chase (FIG. 15C). These studies indicate that the regulation of eIF5A-Hyp stability in islet β cells is different from the other cell types studied to date, and offer one explanation for the relatively rapid inhibition of eIF5A activity in our GC7 inhibition studies.

eIF5A-Hyp is Required for Nuclear Export of Nos2 mRNA

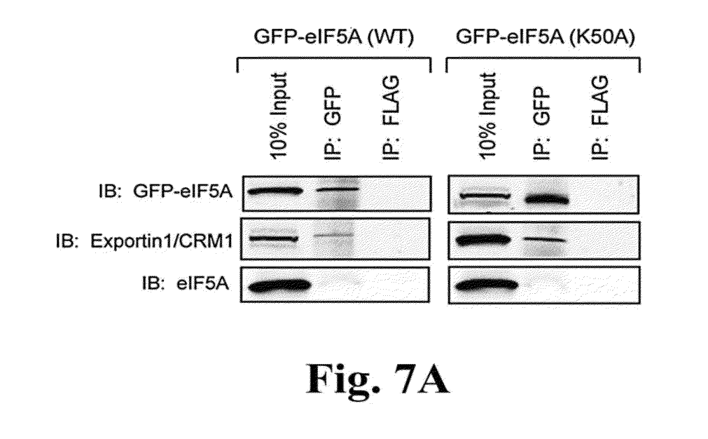

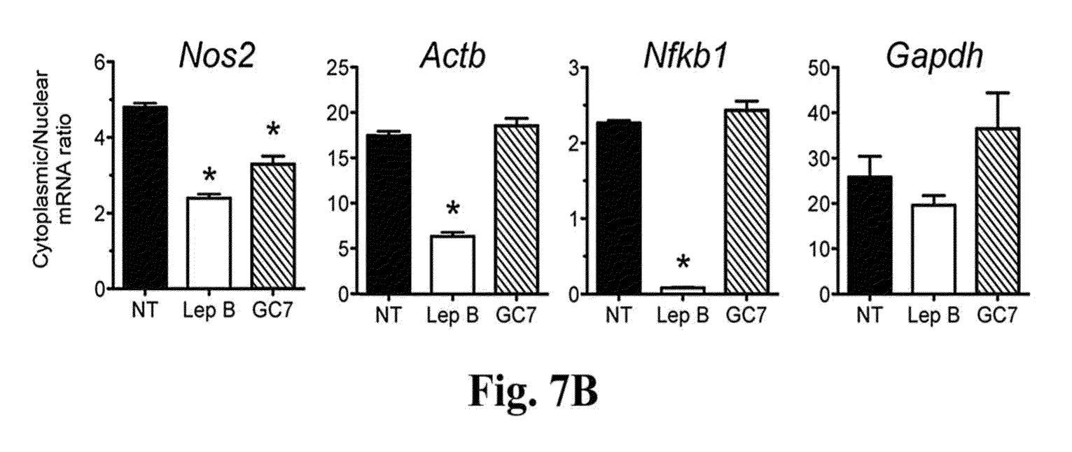

Translational control of mRNA may occur at several levels including, but not limited to, nuclear export and mRNA cycling between polysomes and P bodies/stress granules (reviewed in ref (41). Although some controversy exists in the field regarding eIF5A shuttling (e.g. refs. (42 and 43)), eIF5A has been shown to interact with the nuclear export proteins exportin 1/CRM1 and exportin 4 in mammalian cells (44 and 45), and its interaction with exportin 1/CRM1 in islet β cells (FIG. 7A) is shown in these current studies. Cellular fractionation studies of cytokine-treated INS-1 cells were therefore performed to determine whether eIF5A is necessary for nuclear export of Nos2 mRNA. As shown in FIG. 7B, Nos2, Actb, Nfkb1, and Gapdh mRNAs exhibited a preferentially cytoplasmic distribution in cytokine-treated INS-1 cells. By contrast, preincubation with the exportin 1/CRM1 inhibitor leptomycin B caused a relative retention of Nos2, Actb, and Nfkb1 (but not Gapdh) in the nuclear fraction, indicating that exportin 1/CRM1 serves as a nuclear export protein for these species. Most interestingly, pretreatment of INS-1 cells with GC-7 caused relative nuclear retention of Nos2 mRNA, but not of Actb, Nfkb1, and Gapdh mRNAs (FIG. 7B), indicating that eIF5A-Hyp is required for efficient shuttling of Nos2 mRNA.

Hypusination and Exportin 1/CRM1 are Required for eIF5a Nucleo-Cytoplasmic Shuttling

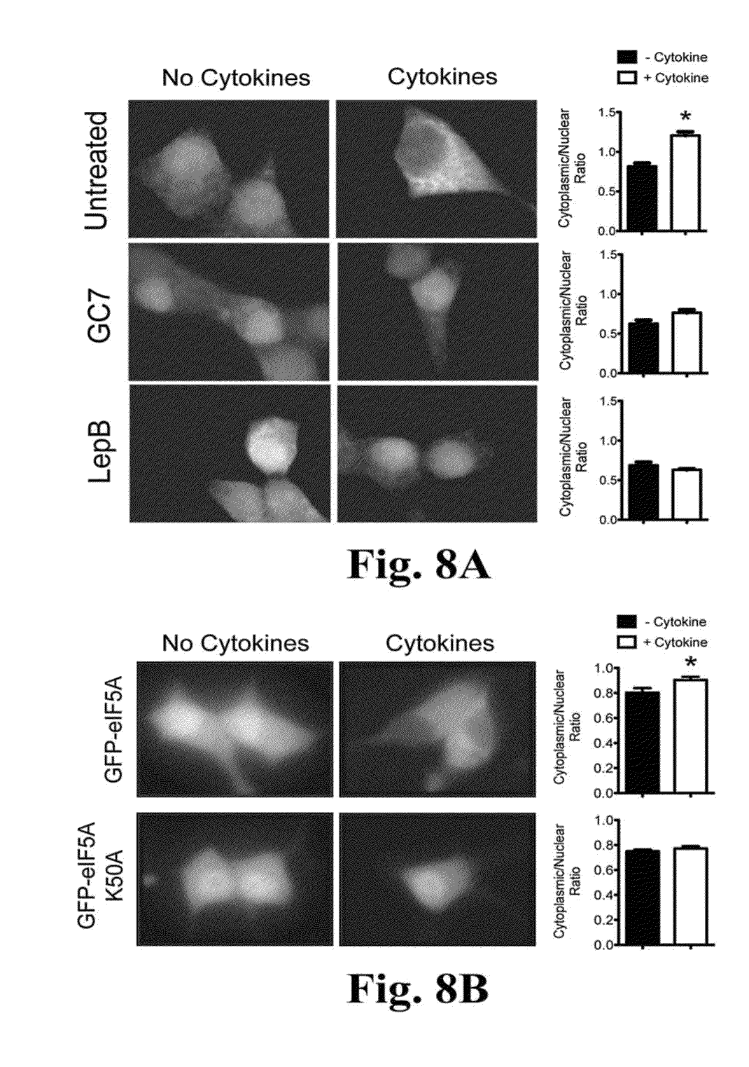

Given the striking retention of Nos2 transcripts in the absence of eIF5A-Hyp, it was of interest to determine whether the intracellular distribution of eIF5A accounts for its effect on Nos2 nucleo-cytoplasmic shuttling. Immunofluorescence of eIF5A in fixed INS-1 cells, were thus performed. As shown in FIG. 8A, eIF5A was found to occupy both cytoplasmic and nuclear distributions in quiescent INS-1 cells (but with a slight nuclear predominance). Upon treatment of cells with cytokines, there was a shift in the eIF5A distribution pattern from the nucleus to the cytoplasm. Interestingly, when cells were pretreated with either GC7 or leptomycin B, treatment with the cytokines failed to induce the nuclear to cytoplasmic translocation of eIF5A. These data indicate that the nuclear-to-cytoplasmic translocation of eIF5A in response to cytokine stimulation is dependent upon both hypusination and the activity of exportin 1/CRM1. To demonstrate that hypusination of Lys50 is required in the nuclear to cytoplasmic translocation of eIF5A, INS-1 cells were transfected with vectors encoding green fluorescent protein (GFP) fusions with either wild-type eIF5A or a mutant eIF5A in which Lys50 is exchanged for Ala (K50A mutant). The images and quantitation in FIG. 8B show that the wild-type fusion protein exhibits nuclear-to-cytoplasmic shuttling upon exposure to cytokines, whereas the localization of the K50A mutant remains unchanged in response to the cytokines Interestingly, the K50A mutant is still observed to interact with exportin 1/CRM1 in co-immunoprecipitation assays (FIG. 7A), consistent with the physical association of eIF5A-Hyp by itself not being sufficient for nuclear-cytoplasmic shuttling. Taken together, these findings support recent studies that demonstrate that hypusination causes a shift in eIF5A localization from the nucleus to the cytoplasm in mammalian cells (46).

eIF5A-Hyp Binds Specifically to Nos2 mRNA

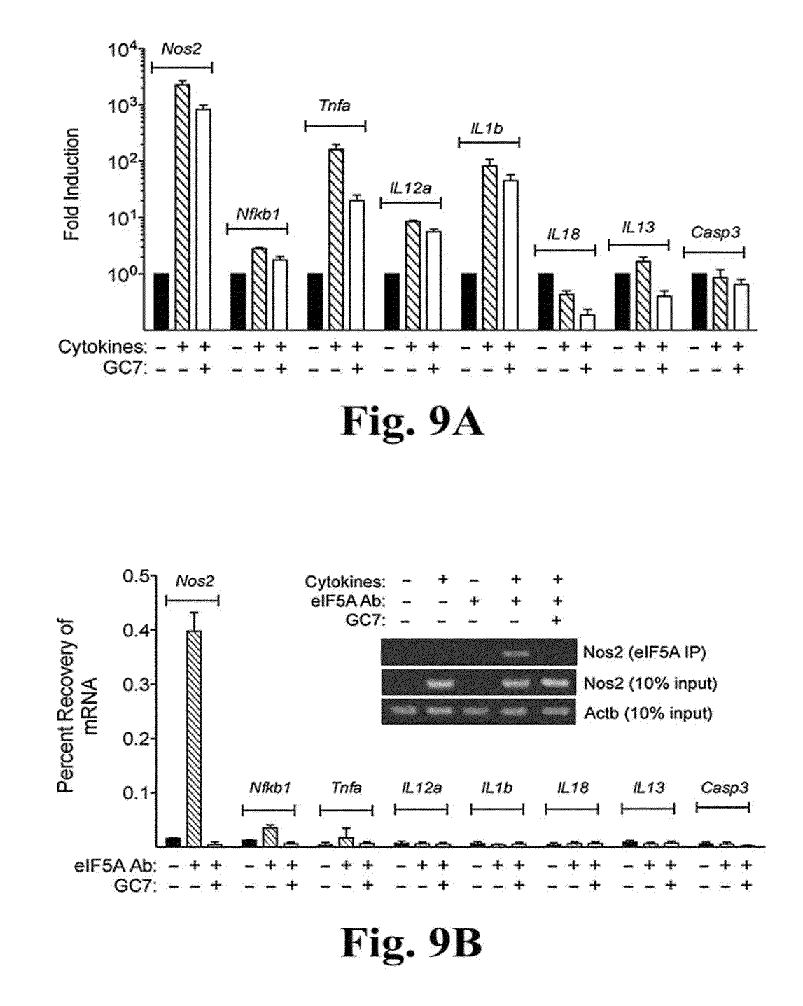

The retention of both Nos2 transcripts and eIF5A in the nucleus upon inhibition of hypusination is consistent with a close relationship between the two molecules, such that eIF5A may serve to chaperone Nos2 transcripts from the nucleus to the cytoplasm in response to cytokine stimulation. eIF5A-Hyp binds to RNAs that contain the consensus sequence 5′-AAAUGU-3′ (47), which is present in the Nos2 mRNA. To determine if eIF5A-Hyp binds Nos2 mRNA, RNA immunoprecipitation assays using INS-1 cell total RNA were performed. As shown in FIG. 9A, cytokine treatment causes induction of a variety of NFkB target genes, including Nos2, Nfkb1, Tnfa, IL12A, and IL1B, but not the induction of non-NFkB targets, including IL18, IL13, and Casp3. Subsequent immunoprecipitation of cytokine-treated INS-1 cells with eIF5A antibody resulted in the co-precipitation of Nos2 transcripts and 10-fold lower, but statistically significant, co-precipitation of Nfkb1 transcripts (FIG. 9B and insert). In contrast, no co-precipitation of other NFkB target and non-target genes was observed. These data document the specificity of mRNA binding by eIF5A-Hyp. When hypusination is blocked by GC7, however, no co-precipitation of any mRNA species is observed, indicating that hypusination of eIF5A is necessary for RNA binding.

### GC7 Treatment Protects Mice Against Streptozotocin (STZ)-Induced Hyperglycemia and Islet Loss

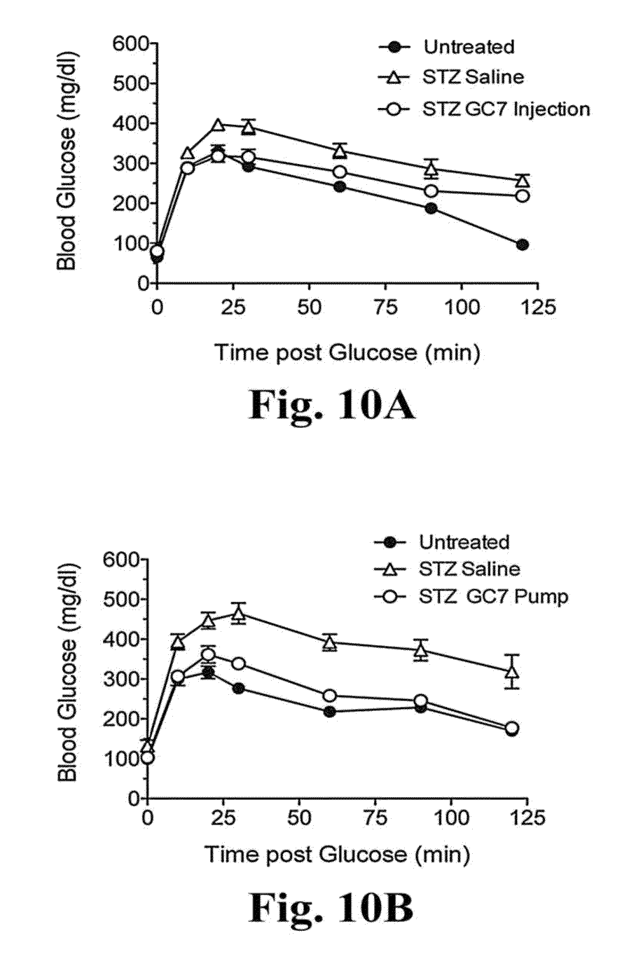

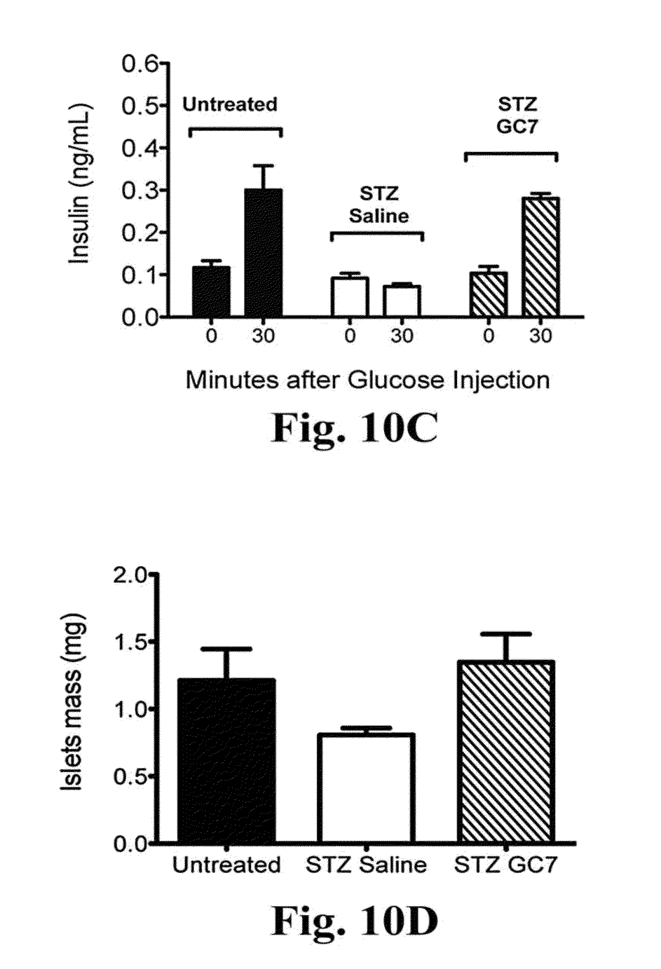

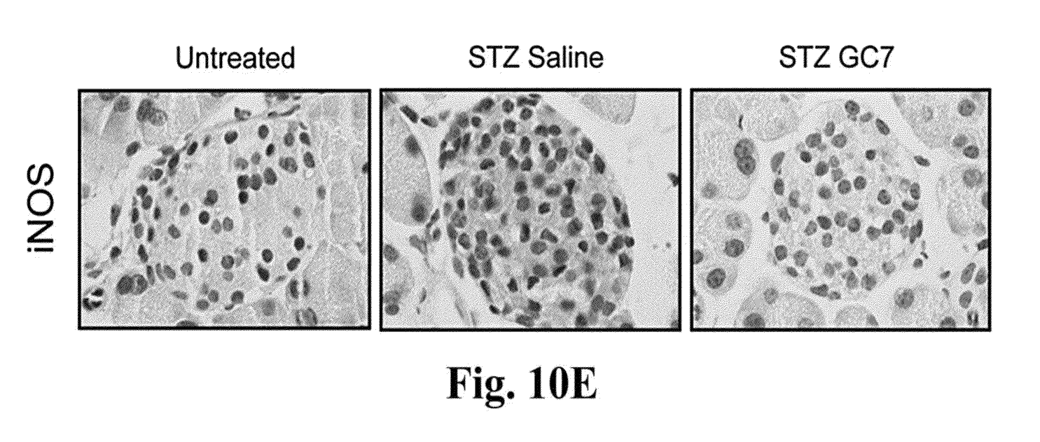

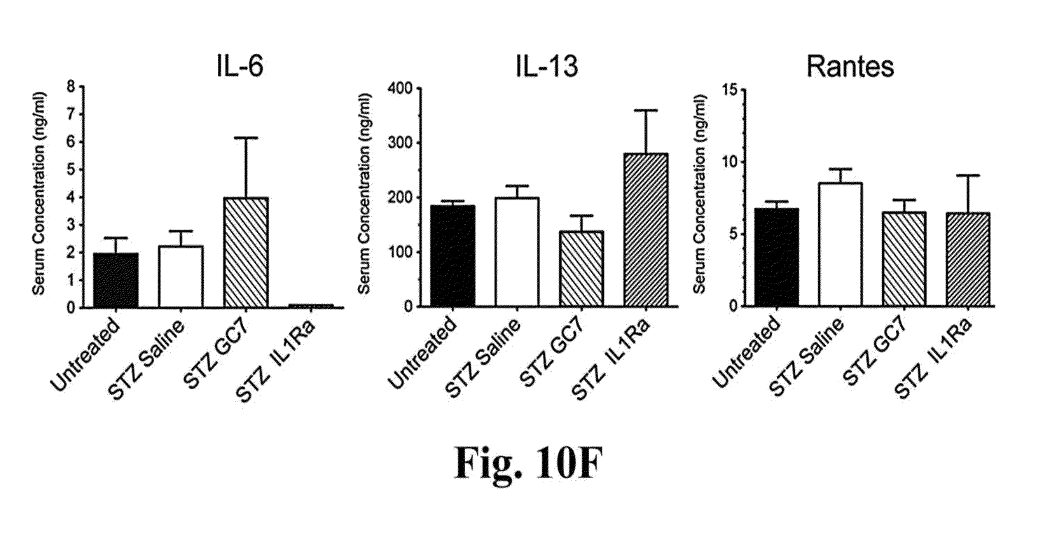

Based on the observed data, if hypusination of eIF5A by DHS is required for cytokine responsiveness in islets, then inhibition of DHS in vivo should protect against low dose STZ diabetes. To test this, C57BL/6J mice were treated with GC7 or vehicle and then subjected to low dose STZ injections. GC7 was delivered in one of two different ways: either by daily bolus intraperitoneal injections (4 mg/kg/day) or by continuous subcutaneous delivery (40 μg/kg/h) using implanted osmotic pumps. A protocol similar to that shown in FIG. 1A was employed in these studies, with GC7 injections or pump implants starting 3 days prior to STZ injections. As shown in FIGS. 10A-E, GC7 treatment by either intraperitoneal injection (FIG. 10A) or osmotic pump (FIG. 10B) led to near-complete protection of animals from STZ-induced glucose intolerance.

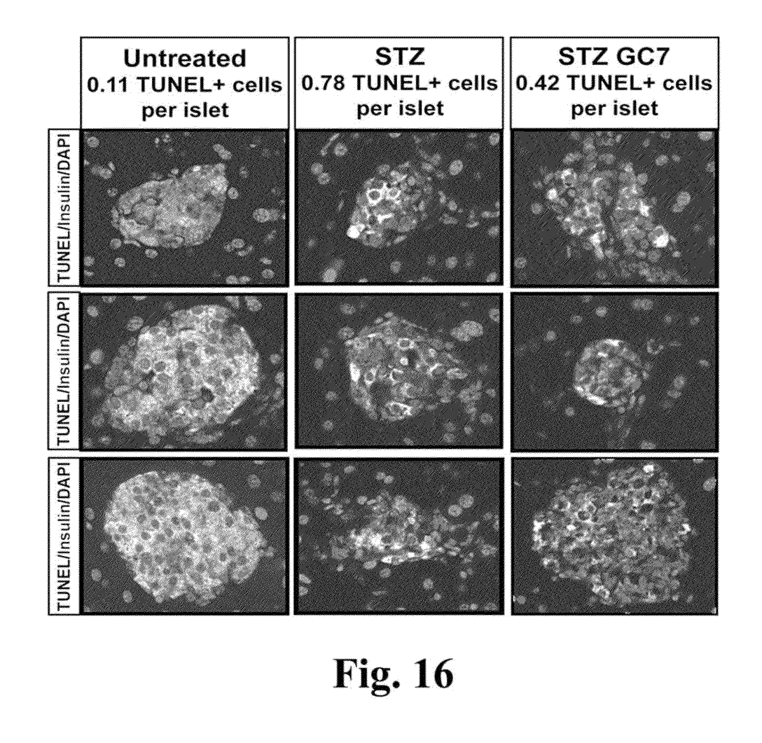

Insulin levels obtained at 0 and 30 min. during glucose challenge of pump-implanted animals revealed a defect in insulin secretion in vehicle treated STZ animals, whereas GC7-treated STZ animals exhibited a normal insulin secretory response (FIG. 10C) consistent with β cell preservation. Histomorphometric analysis of pancreata revealed a trend to reduced β cell mass in control STZ animals (p=0.083), with full preservation of mass in GC7-injected animals (FIG. 10D). As with si-eIF5A treatment, islets of GC7-treated pump animals showed suppression of iNOS production (FIG. 10E). Consistent with the known β cell toxic effects of STZ, analysis of pancreatic sections from these animals revealed an increase in β cell TUNEL-positivity with STZ treatment, with GC7-treated animals showing fewer TUNEL+ cells per islet (0.42) compared to STZ treatment alone (0.78) (FIG. 16). These data therefore are consistent with eIF5A-Hyp playing an essential role in the early events that lead to islet dysfunction and death in response to inflammation.

eIF5A-Hyp Promotes Islet Inflammation Independently of the Immune System

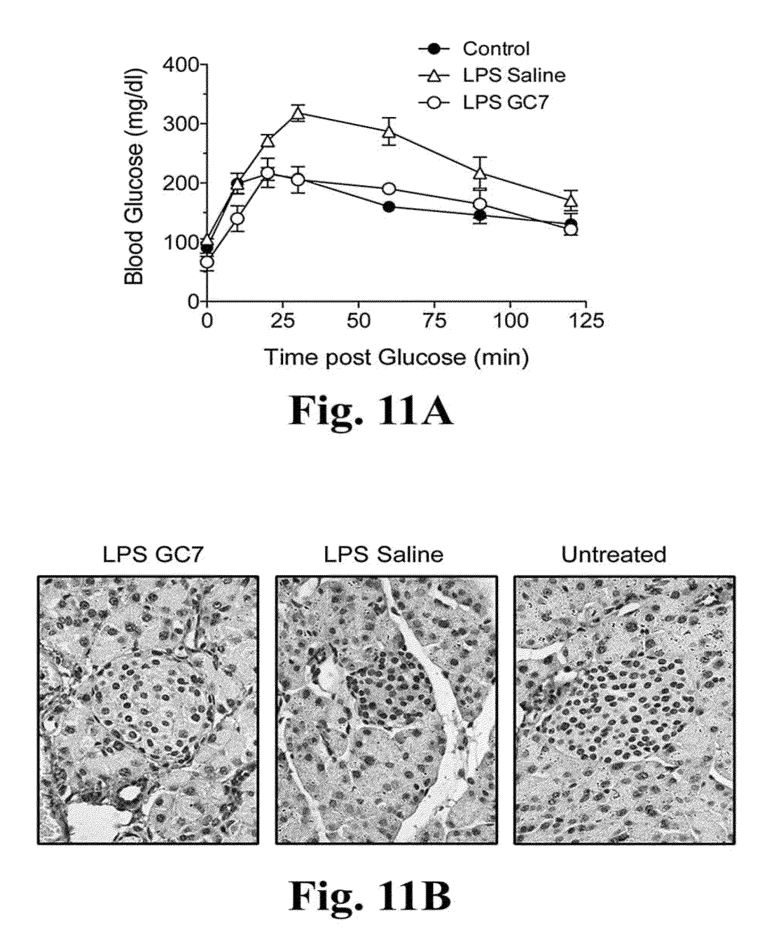

Because systemic GC7 delivery would be expected to inhibit hypusination in all cells that express DHS, it is unclear if the islet protection afforded by GC7 in vivo is a result of its effects in the islet, the immune cells, or both. In order to clarify this, the IL-1 responses in STZ-treated C57BL/6J animals were assessed by measuring levels of the IL-1-responsive cytokine IL-6. As shown in FIG. 10F, whereas concurrent treatment with STZ and IL-1R^{a} caused a dramatic drop in serum IL-6 levels (consistent with IL-1 inhibition), concurrent treatment with STZ and GC7 did not affect IL-6 levels. No alterations in serum levels of IL13 and Rantes/CCL5 were observed between treatment groups (FIG. 10F). This result indicated that the protection by DHS inhibition was not simply a consequence of inhibiting systemic IL-1 release. To address the role of immune cells more directly, a mouse model was generated to test the role of hypusination in the islet inflammatory response independently of the immune system. Lipopolysaccharide (LPS) is an agent that evokes the NFkB response through activation of the toll-like receptor 4 (TLR4) (48), which is also expressed in pancreatic islets (49). Immune-deficient NOD/Scid-(IL-2Rγ-null) mice were therefore injected with a single injection of LPS (20 mg/kg, a dose that is known to cause massive inflammatory responses in immune-competent mice, ref (21)) concurrently with either GC7 (4 mg/kg/day intraperitoneally) or vehicle. IPGTTs performed 3 days following the LPS injection revealed glucose intolerance in vehicle-treated mice compared to non-LPS controls (FIG. 11A). Interestingly, GC7-treated mice that received LPS showed no evidence of glucose intolerance (FIG. 11A). Immunohistochemical analysis of pancreata revealed increased iNOS staining intensity in islets of vehicle-treated animals, but not in GC7-treated animals (FIG. 11B). These data indicate that inhibition of hypusination in vivo can protect islets from iNOS-mediated dysfunction independently of the immune system, and that hypusination occurs within the islet.

### Materials and Methods

Animals and cells: C57BL/6J mice were purchased from Jackson Labs, and NOD/Scid-(IL-2Rγ-null) mice were bred at the Indiana University Simon Cancer Center. All animal studies were performed under protocols approved by the Indiana University School of Medicine Animal Care and Use Committee or the University of Virginia Animal Care and Use Committee. The cytokine-responsive rat insulinoma cell line INS-1 (832/13) was maintained as previously described (68), and transfected using Metafectene™ Pro (Biontex) according to manufacturer's instructions. C57BL/6J mouse islets were isolated from collagenase-digested pancreata as described (69 and 70), then handpicked and cultured in RPMI medium overnight prior to use. Human islets were obtained commercially (Beta-Pro).

Antibodies and vectors: Polyclonal antibody against eIF5A was from Abcam, and monoclonal antibody against eIF5A was from BD Bioscience; monoclonal antibody against eIF5A2 was from Abnova; monoclonal antibody against FLAG-M2 was from Sigma; monoclonal anti-GFP antibody was from Abgent; monoclonal antibody against actin (clone C4) was from MP Biomedicals; anti-iNOS polyclonal antibody was from Millipore. For immunoblots, fluorophore-labeled secondary antibodies were from Li-Cor (IRDye 800 and IRDye 700). The cDNAs encoding eIF5A1 and eIF5A2 were obtained by PCR cloning from reverse-transcribed human islet RNA, then subcloned into the cytomegalovirus promoter-driven vector pEGFP (Clontech), and verified by automated sequencing. The K50A mutation of eIF5A was generated using oligonucleotide-directed mutagenesis.

Small interfering RNA (siRNA) studies: Stabilized siRNAs for intraperitoneal injections were custom synthesized by Dharmacon using the siSTABLE® modification. Groups of 10 week-old C57BL/6J male mice (from Jackson Labs) received daily intraperitoneal injections of 1.6 mg/kg siRNA prepared in 0.9% saline or vehicle alone (0.9% saline) for 3 days. For in vitro studies, islets from each group of injected mice were harvested on the fourth day and pooled prior to analysis. Injections with each siRNA were performed on at least 3 different occasions. siRNA sequences were as follows: siControl, 5′-AAAGUCGACCUUCAGUAAGGA-3′; si-eIF5A, 5′-AACGGAAUGACUUCCAGCUGA-3′. For siRNA studies in the rat β cell-derived line INS-1 (832/13), cells were transfected with a SMART Pool® siRNA against rat eIF5A1 (Dharmacon, #L-083855-01) or non-targeting control siRNA #1 (Dharmacon) using DharmaFECT® transfection reagent (Dharmacon).

Cytokine and inhibitor incubation studies: For cytokine incubation assays, a 1000× cocktail of cytokines containing 5 μg/ml IL1β, 10 μg/ml TNFα, and 100 μg/ml IFNγ (all prepared in 0.1% BSA in Tris-buffered saline) was applied at 1× final concentration to groups of 50 islets or 1×10⁶ INS-1 (832/13) cells for a total of 4 h at 37° C. For GC7 incubation studies, GC7 was prepared at a stock concentration of 125 mM in 10 mM acetic acid and applied to cultures of 50 islets or 1×10⁶ INS-1 cells to obtain the final concentrations indicted in the figures. Where needed, GC7 was protected from amine oxidases in serum by addition of 1 mM aminoguanidine (Sigma). Cells were incubated with GC7 overnight (˜16 h) at 37° C. prior to analysis. Cells were exposed to Leptomycin B (Cayman Chemicals) for 3 hours; leptomycin B was prepared at a 1000× stock concentration of 20 μg/ml in ethanol.

For flow cytometry studies, INS-1 cells (serum starved or pretreated with GC7 as indicated in the figures) were stained with calcein-AM and ethidium homodimer 1 (Live/Dead® kit, Invitrogen) for 30 minutes, and 30,000 cells/sample were analyzed for green (living cells) and red (dead cells) fluorescence using a FACS Calibur (BD bioscience) instrument. For cell cycle analysis, 10⁶ cells were washed in PBS and fixed in ice-cold 70% ethanol for 1 hour. After washing in PBS, cells were re-suspended in Guava cell cycle reagent (Millipore) and incubated at room temperature for 30 minutes. Intercalation of propidium iodide into cellular DNA was quantitated using a FACS Calibur instrument and the data analyzed for cell cycle status using Modfit software.

Immunofluorescence: Immunofluorescence of INS-1 cells proceeded essentially as described previously (71), using primary antibodies to eIF5A1 and secondary anti-mouse Alexa-488-conjugated antibody (Invitrogen), or by direct visualization of GFP fusion proteins at 488 nm. Cells were counterstained with 4′,6-diamidino-2-phenylindole (DAPI) to visualize nuclei, then imaged using an Axio-Observer Z1 (Zeiss) inverted fluorescent microscope equipped with an Orca ER CCD camera (Hammamatsu). Quantitation of cytoplasic-to-nuclear ratios of eIF5A staining was performed using Axio-Vision Software, v. 4.7 (Zeiss).

Immunostaining and morphometric assessment of β cell mass: Immunostaining of pancreatic sections proceeded as described previously (72). For assessment of islet cell death in pancreatic sections, the technique of terminal deoxynucleotidyl transferase dUTP nick end labeling (TUNEL) was performed using biotinylated 16-dUTP (Roche) and Texas Red Neutravidin (Invitrogen). Digital images of each islet at 40× magnification were acquired using an Axio-Observer D1 microscope (Zeiss) inverted fluorescent microscope equipped with a high-resolution color camera. TUNEL-positive/insulin-positive nuclei were counted manually by an observer blinded to sample identity, and data were re-coded as the average number of TUNEL-positive nuclei per islet. β cell mass was calculated as described previously (73), but with some modifications. Briefly, pancreata from 3 mice per treatment group were rapidly dissected and weighed, fixed in 4% paraformaldehyde, paraffin-embedded, and longitudinally sectioned. Three sections/pancreas (approx. 75 μm apart) were subsequently immunostained for insulin and counterstained with hematoxylin as described (74), and digital images of each section at 10× magnification were acquired on an Axio-Observer Z1 microscope (Zeiss) fitted with an AxioCam high resolution color camera. Relative β cell area (calculated using Axio-Vision Software) was multiplied by pancreatic weight to obtain β cell mass. Data represent the average from three sections per pancreas, and 3 pancreata from each treatment group.

RNA immunoprecipitation (RIP) assays: RIP assays from 1×10⁷ formaldehyde cross-linked INS-1 cells were performed as described (75). Isotype-matched antibodies against eIF5A and the FLAG-M2 epitope (for control immunoprecipitations) were used at a final dilution of 1:100. Immunoprecipitated RNA was reversed transcribed and subjected to quantitative PCR amplification for selected genes as described above. All data represent the average of triplicate determinations from at least 3 independent RIP assays.

Immunoblot and nitrite and iNOS assays: Whole cell extracts were resolved by electrophoresis on a 4-20% SDS-polyacrylamide gel, followed by immunoblot using anti-eIF5A1, anti-eIF5A2, anti-iNOS, anti-GFP, or anti-actin primary antibodies and fluorophore-labeled secondary antibodies. Immunoblots were visualized using the Li-Cor® Odyssey® system (Li-Cor Biosciences) and quantitated by scanning fluorometry using Odyssey Imaging v. 3.0 software (Li-Cor Biosciences). Nitrite was quantitated by measuring nitric oxide-derived nitrite from INS-1 cell culture medium using the Griess reagent (Promega) according to the manufacturer's recommendations. iNOS activity was measured using a commercially available kit (Cayman).

Subcellular fractionation studies: Nuclear and cytoplasmic fractions of 1×10⁶ cytokine-treated INS-1 cells exposed to vehicle, leptomycin B (20 ng/ml), or GC7 (100 μM) were prepared using the method of Dignam, et al. (76). Total RNA was isolated from nuclear and cytoplasmic fractions using the RNeasy® RNA isolation kit (Qiagen).

Quantitative RT-PCR: Five micrograms of total RNA from islets or INS-1 cells were reverse-transcribed as detailed previously (77). cDNA was subjected to quantitative PCR using SYBR Green-based technology and published primers for mouse Ins1/2 pre-mRNA (78) and QuantiTech® primers (Qiagen) for all other mouse genes. The Assay on Demand® kit (Applied Biosystems) was used to amplify rat genes in INS-1 cells and human genes in human islets. Thermal cycling was performed according to manufacturer's instructions, and the identity of each PCR product was verified by automated sequencing. All samples were corrected for total input RNA as quantitated by an Experion® (Bio-Rad) bioanalyzer. All data represent the average of triplicate determinations from at least 3 independent experiments.

GSCa imaging assays: Intracellular Ca²⁺ ((Ca2+)ᵢ) was measured using the ratiometric Ca²⁺ indicator fura-2 AM using a modification of previously published methods (79). The glucose-stimulated (Ca2+)ᵢ response (GSCa) was defined as the difference between ratio measurements (340/380 nm fluorescence) in 11 mM vs. 3 mM glucose. Data were analyzed with IP Lab's software version 4.0 (Scanalytics).

GSIS and IPGTT studies: For GSIS studies using isolated islets, approximately 50 islets or 1×10⁶ INS-1 cells per condition were washed and incubated in Krebs-Ringer HEPES-buffered (KRB) solution for one hour at 37° C., and then placed in KRB solution containing 3 mM or 11 mM glucose for one hour. Insulin released into the medium was assayed using a two-site immunospecific ELISA (ALPCO Diagnostics). All data represent the average of 3 independent experiments. IPGTTs in mice were performed after an overnight fast. Blood glucose was measured from samples taken from the tail vein before intraperitoneal injection of 1 g glucose per kg mouse weight, and at 10, 20, 30, 60, 90, 120, and 180 minutes after glucose administration using an A1phaTrak® glucometer (Abbott).

Studies in vivo: Daily STZ injections to groups of 10 week-old C57BL/6J and NOD/Scid-(IL-2Rγ-null) mice proceeded as described previously (30) at a dose of 55 mg STZ per kg mouse weight for 5 days. GC7 was administered by either daily intraperitoneal injection at a dose of 4 mg/kg mouse weight throughout the duration of the study, or by continuous delivery (40 μg/kg/hour) via a subcutaneously implanted osmotic pump (Alzet). The IL-1Ra anakinara (50 mg/kg) was given 30 min prior to the first dose of STZ, followed by twice daily doses (at 20 mg/kg) until the end of the study. LPS (at a single dose of 20 mg/kg) was given to NOD/Scid-(IL-2Rγ-null) mice by intraperitoneal injection, with or without co-treatment with GC7 as described above. For measurement of serum cytokines, serum was collected via cardiac puncture at the time of euthanasia. Serum was analyzed using a Luminex system (Millipore) and a mouse cytokine/chemokines Panel 1 mutliplex kit (Millipore).

Measurement of hvpusination: Approximately 100 islets or 1×10⁶ INS-1 cells per condition were incubated with 1.5 μCi ³H-spermidine (Perkin Elmer) in the presence of 1 mM aminoguanidine. The measurement of eIF5A-Hyp half-life proceeded as described previously (36) with some modification. Briefly, following a 4 h preincubation with ³H-spermidine, INS-1 cells or islets were incubated with 1 mM spermidine plus 1 mM aminoguanidine for various times, then whole-cell extracts were isolated and subjected to electrophoresis on a 12% SDS-polyacrylamide gel. Gels were visualized by fluorography, and bands were quantitated using Kodak Molecular Imaging Software v. 5.0 (Kodak).

Statistics: Sample statistics were calculated using repeated measures ANOVA, one-way ANOVA, and Student's t test as indicated in the figure legends.

### Federally Registered Trademarks:

The owners of trademarks designated herein are provided below:
“siSTABLE” is a registered trademark of DHARMACON INC. CORPORATION DELAWARE 1376 Miners Drive, #101 Lafayette COLORADO 80026.
2. “SMART Pool” is a registered trademark of Dharmacon Research CORPORATION COLORADO 1376 Miners Drive, #101 Lafayette COLORADO 80026.
3. “DharmaFECT” is a registered trademark of DHARMACON INC. CORPORATION DELAWARE 2650 Crescent Drive, #100 Lafayette COLORADO 80026.
4. “Live/Dead” is a registered trademark of MOLECULAR PROBES, INC. CORPORATION OREGON 4849 Pitchford Avenue Eugene Oreg. 974029144.
5. “Li-Cor” is a registered trademark of Li-Cor, Inc. CORPORATION NEBRASKA 4421 Superior St. Lincoln NEBRASKA 68504.
6. “Odyssey” is a registered trademark of LI-COR, Inc. CORPORATION NEBRASKA 4647 Superior Street Lincoln Nebr. 68504.
7. “RNeasy” is a registered trademark of Qiagen GmbH CORPORATION FED REP GERMANY Max-Volmer-Str. 4 D-40724 Hilden FED REP GERMANY.
8. “QuantiTech” is a registered trademark of QuantiTech, Inc. CORPORATION ALABAMA 300 Voyager Way Ste. 300 Huntsville ALABAMA 35806.
9. “Assay on Demand” is a registered trademark of Applera Corporation DELAWARE 850 Lincoln Centre Drive Foster City Calif. 94404.
10. “Experion” is a registered trademark of Honeywell International Inc. CORPORATION DELAWARE 101 Columbia Road Morristown NEW JERSEY 07962.
11. “Alpha Trak” is a registered trademark of Alpha Environmental Management Corp, LLC LIMITED LIABILITY COMPANY FLORIDA 1340 Tuskawilla Road, Suite 113 Winter Springs FLORIDA 32708.

### Summary:

The present invention contemplates modifications as would occur to those skilled in the art. It is also contemplated that additional agents beyond those specifically disclosed herein which are capable of impairing the translation of mRNA encoding inducible nitric oxide synthase within a pancreatic islet or an agent capable of interfering with the hypusination of eIF5A thereby furthering the survival of pancreatic islets are embodied herein without departing from the spirit of the present disclosure. All publications cited in this specification are herein incorporated by reference as if each individual publication, patent, or patent application was specifically and individually indicated to be incorporated by reference and set forth in its entirety herein.

Further, any theory of operation, proof, or finding stated herein is meant to further enhance understanding of the present invention and is not intended to make the scope of the present invention dependent upon such theory, proof, or finding. While the invention has been illustrated and described in detail in the figures and foregoing description, the same is considered to be illustrative and not restrictive in character, it being understood that only the preferred embodiments have been shown and described and that all changes and modifications that come within the spirit of the invention are desired to be protected.

### REFERENCES

1. Prentki, M, and Nolan, C J. Islet beta cell failure in type 2 diabetes. J Clin Invest. 2006; 116(7):1802-1812.
2. Mathis, D, Vence, L, and Benoist, C. beta-Cell death during progression to diabetes. Nature. 2001; 414(6865):792-798.
3. Spranger, J, Kroke, A, Mohlig, M, Hoffmann, K, Bergmann, M M, Ristow, M, Boeing, H, and Pfeiffer, A F. Inflammatory cytokines and the risk to develop type 2 diabetes: results of the prospective population-based European Prospective Investigation into Cancer and Nutrition (EPIC)-Potsdam Study. Diabetes. 2003; 52(3):812-817.
4. Sauter, N S, Schulthess, F T, Galasso, R, Castellani, L W, and Maedler, K. The antiinflammatory cytokine interleukin-1 receptor antagonist protects from high-fat diet-induced hyperglycemia. Endocrinology. 2008; 149(5):2208-2218.
5. Larsen, C M, Faulenbach, M, Vaag, A, Volund, A, Ehses, J A, Seifert, B, Mandrup-Poulsen, T, and Donath, M Y. Interleukin-1-receptor antagonist in type 2 diabetes mellitus. N Engl J. Med. 2007; 356(15):1517-1526.
6. Eizirik, D L, and Mandrup-Poulsen, T. A choice of death—the signal-transduction of immune-mediated beta-cell apoptosis. Diabetologia. 2001; 44(12):2115-2133.
7. Leist, M, Single, B, Naumann, H, Fava, E, Simon, B, Kuhnle, S, and Nicotera, P. Inhibition of mitochondrial ATP generation by nitric oxide switches apoptosis to necrosis. Exp Cell Res. 1999; 249(2):396-403.
8. Koster, J C, Marshall, B A, Ensor, N, Corbett, J A, and Nichols, C G. Targeted overactivity of beta cell K(ATP) channels induces profound neonatal diabetes. Cell. 2000; 100(6):645-654.
9. Chambers, K T, Unverferth, J A, Weber, S M, Wek, R C, Urano, F, and Corbett, J A. The role of nitric oxide and the unfolded protein response in cytokine-induced beta-cell death. Diabetes. 2008; 57(1):124-132.
10. Collier, J J, Fueger, P T, Hohmeier, H E, and Newgard, C B. Pro- and antiapoptotic proteins regulate apoptosis but do not protect against cytokine-mediated cytotoxicity in rat islets and beta-cell lines. Diabetes. 2006; 55(5):1398-1406.
11. Larsen, L, Tonnesen, M, Ronn, S G, Storling, J, Jorgensen, S, Mascagni, P, Dinarello, C A, Billestrup, N, and Mandrup-Poulsen, T. Inhibition of histone deacetylases prevents cytokine-induced toxicity in beta cells. Diabetologia. 2007; 50(4):779-789.
12. Steer, S A, Scarim, A L, Chambers, K T, and Corbett, J A. Interleukin-1 stimulates beta-cell necrosis and release of the immunological adjuvant HMGB1. PLoS Med. 2006; 3(2):e17.
13. Facchiano, A M, Stiuso, P, Chiusano, M L, Caraglia, M, Giuberti, G, Marra, M, Abbruzzese, A, and Colonna, G. Homology modelling of the human eukaryotic initiation factor 5A (eIF-5A). Protein Eng. 2001; 14(11):881-890.
14. Cooper, H L, Park, M H, Folk, J E, Safer, B, and Braverman, R. Identification of the hypusine-containing protein hy+ as translation initiation factor eIF-4D. Proc Natl Acad Sci USA. 1983; 80(7):1854-1857.
15. Park, M H, Nishimura, K, Zanelli, C F, and Valentini, S R. Functional significance of eIF5A and its hypusine modification in eukaryotes. Amino Acids. 2009.
16. Park, M H, Wolff, E C, Lee, Y B, and Folk, J E. Antiproliferative effects of inhibitors of deoxyhypusine synthase. Inhibition of growth of Chinese hamster ovary cells by guanyl diamines. J Biol. Chem. 1994; 269(45):27827-27832.
17. Caraglia, M, Marra, M, Giuberti, G, D'Alessandro, A M, Baldi, A, Tassone, P, Venuta, S, Tagliaferri, P, and Abbruzzese, A. The eukaryotic initiation factor 5A is involved in the regulation of proliferation and apoptosis induced by interferon-alpha and EGF in human cancer cells. J Biochem. 2003; 133(6):757-765.
18. Taylor, C A, Senchyna, M, Flanagan, J, Joyce, E M, Cliche, D O, Boone, A N, Culp-Stewart, S, and Thompson, J E. Role of eIF5A in TNF-alpha-mediated apoptosis of lamina cribrosa cells. Invest Ophthalmol Vis Sci. 2004; 45(10):3568-3576.
19. Taylor, C A, Sun, Z, Cliche, D O, Ming, H, Eshaque, B, Jin, S, Hopkins, M T, That, B, and Thompson, J E. Eukaryotic translation initiation factor 5A induces apoptosis in colon cancer cells and associates with the nucleus in response to tumour necrosis factor alpha signalling. Exp Cell Res. 2007; 313(3):437-449.
20. Li, A L, Li, H Y, Jin, B F, Ye, Q N, Zhou, T, Yu, XD, Pan, X, Man, J H, He, K, Yu, M, Hu, M R, Wang, J, Yang, S C, Shen, B F, and Zhang, X M. A novel eIF5A complex functions as a regulator of p53 and p53-dependent apoptosis. J Biol. Chem. 2004; 279(47):49251-49258.
21. Moore, C C, Martin, E N, Lee, G, Taylor, C, Dondero, R, Reznikov, L L, Dinarello, C, Thompson, J, and Scheld, W M. Eukaryotic Translation Initiation Factor 5A Small Interference RNA-Liposome Complexes Reduce Inflammation and Increase Survival in Murine Models of Severe Sepsis and Acute Lung Injury. J Infect Dis. 2008; 198(9):1407-1414.
22. Maier, L M, and Wicker, L S. Genetic susceptibility to type 1 diabetes. Curr Opin Immunol. 2005; 17(6):601-608.
23. Grattan, M, Mi, Q S, Meagher, C, and Delovitch, T L. Congenic mapping of the diabetogenic locus Idd4 to a 5.2-cM region of chromosome 11 in NOD mice: identification of two potential candidate subloci. Diabetes. 2002; 51(1):215-223.
24. Kruse, M, Rosorius, O, Kratzer, F, Bevec, D, Kuhnt, C, Steinkasserer, A, Schuler, G, and Hauber, J. Inhibition of CD83 cell surface expression during dendritic cell maturation by interference with nuclear export of CD83 mRNA. J Exp Med. 2000; 191(9):1581-1590.
25. Clement, P M, Johansson, H E, Wolff, E C, and Park, M H. Differential expression of eIF5A-1 and eIF5A-2 in human cancer cells. FEBS J. 2006; 273(6):1102-1114.
26. Jenkins, Z A, Haag, P G, and Johansson, H E. Human eIF5A2 on chromosome 3q25-q27 is a phylogenetically conserved vertebrate variant of eukaryotic translation initiation factor 5A with tissue-specific expression. Genomics. 2001; 71(1):101-109.
27. Lukic, M L, Stosic-Grujicic, S, and Shahin, A. Effector mechanisms in low-dose streptozotocin-induced diabetes. Dev Immunol. 1998; 6(1-2):119-128.
28. Calderon, B, Suri, A, Miller, M J, and Unanue, E R. Dendritic cells in islets of Langerhans constitutively present beta cell-derived peptides bound to their class II MHC molecules. Proc Natl Acad Sci USA. 2008; 105(16):6121-6126.
29. Lenzen, S. The mechanisms of alloxan- and streptozotocin-induced diabetes. Diabetologia. 2008; 51(2):216-226.
30. Flodstrom, M, Tyrberg, B, Eizirik, D L, and Sandler, S. Reduced sensitivity of inducible nitric oxide synthase-deficient mice to multiple low-dose streptozotocin-induced diabetes. Diabetes. 1999; 48(4):706-713.
31. Henquin, J C, Nenquin, M, Stiernet, P, and Ahren, B. In vivo and in vitro glucose-induced biphasic insulin secretion in the mouse: pattern and role of cytoplasmic Ca2+ and amplification signals in beta-cells. Diabetes. 2006; 55(2):441-451.
32. Kolb, H, and Mandrup-Poulsen, T. An immune origin of type 2 diabetes? Diabetologia. 2005; 48(6):1038-1050.
33. Corbett, J A, and McDaniel, M L. Intraislet release of interleukin 1 inhibits beta cell function by inducing beta cell expression of inducible nitric oxide synthase. J Exp Med. 1995; 181(2):559-568.
34. Chen, K Y, and Liu, A Y. Biochemistry and function of hypusine formation on eukaryotic initiation factor 5A. Biol Signals. 1997; 6(3):105-109.
35. Dong, Z, Arnold, R J, Yang, Y, Park, M H, Hrncirova, P, Mechref, Y, Novotny, M V, and Zhang, J T. Modulation of differentiation-related gene 1 expression by cell cycle blocker mimosine, revealed by proteomic analysis. Mol Cell Proteomics. 2005; 4(7):993-1001.
36. Nishimura, K, Murozumi, K, Shirahata, A, Park, M H, Kashiwagi, K, and Igarashi, K. Independent roles of eIF5A and polyamines in cell proliferation. Biochem J. 2005; 385(Pt 3):779-785.
37. Corbett, J A, and McDaniel, M L. The Use of Aminoguanidine, a Selective iNOS Inhibitor, to Evaluate the Role of Nitric Oxide in the Development of Autoimmune Diabetes. Methods. 1996; 10(1):21-30.
38. Herbst, M, Sasse, P, Greger, R, Yu, H, Hescheler, J, and Ullrich, S. Membrane potential dependent modulations of calcium oscillations in insulin-secreting INS-1 cells. Cell Calcium. 2002; 31(3):115-126.
39. Duncan, R F, and Hershey, J W. Changes in eIF-4D hypusine modification or abundance are not correlated with translational repression in HeLa cells. J Biol. Chem. 1986; 261(27):12903-12906.
40. Gerner, E W, Mamont, P S, Bernhardt, A, and Siat, M. Post-translational modification of the protein-synthesis initiation factor eIF-4D by spermidine in rat hepatoma cells. Biochem J. 1986; 239(2):379-386.
41. Parker, R, and Sheth, U. P bodies and the control of mRNA translation and degradation. Mol. Cell. 2007; 25(5):635-646.
42. Jao, D L, and Yu Chen, K. Subcellular localization of the hypusine-containing eukaryotic initiation factor 5A by immunofluorescent staining and green fluorescent protein tagging. J Cell Biochem. 2002; 86(3):590-600.
43. Shi, X P, Yin, K C, Zimolo, Z A, Stern, A M, and Waxman, L. The subcellular distribution of eukaryotic translation initiation factor, eIF-5A, in cultured cells. Exp Cell Res. 1996; 225(2):348-356.
44. Rosorius, O, Reichart, B, Kratzer, F, Heger, P, Dabauvalle, M C, and Hauber, J. Nuclear pore localization and nucleocytoplasmic transport of eIF-5A: evidence for direct interaction with the export receptor CRM1. J Cell Sci. 1999; 112 (Pt 14):2369-2380.
45. Lipowsky, G, Bischoff, F R, Schwarzmaier, P, Kraft, R, Kostka, S, Hartmann, E, Kutay, U, and Gorlich, D. Exportin 4: a mediator of a novel nuclear export pathway in higher eukaryotes. EMBO J. 2000; 19(16):4362-4371.
46. Lee, S B, Park, J H, Kaevel, J, Sramkova, M, Weigert, R, and Park, M H. The effect of hypusine modification on the intracellular localization of eIF5A. Biochem Biophys Res Commun. 2009; 383(4):497-502.
47. Xu, A, and Chen, K Y. Hypusine is required for a sequence-specific interaction of eukaryotic initiation factor 5A with postsystematic evolution of ligands by exponential enrichment RNA. J Biol. Chem. 2001; 276(4):2555-2561.
48. Chow, J C, Young, D W, Golenbock, D T, Christ, W J, and Gusovsky, F. Toll-like receptor-4 mediates lipopolysaccharide-induced signal transduction. J Biol. Chem. 1999; 274(16):10689-10692.
49. Schulthess, F T, Paroni, F, Sauter, N S, Shu, L, Ribaux, P, Haataja, L, Strider, R M, Oberholzer, J, King, C C, and Maedler, K. CXCL10 impairs beta cell function and viability in diabetes through TLR4 signaling. Cell Metab. 2009; 9(2):125-139.
50. Kemper, W M, Berry, K W, and Merrick, W C. Purification and properties of rabbit reticulocyte protein synthesis initiation factors M2Balpha and M2Bbeta. J Biol. Chem. 1976; 251(18):5551-5557.
51. Saini, P, Eyler, D E, Green, R, and Dever, T E. Hypusine-containing protein eIF5A promotes translation elongation. Nature. 2009; 459(7243):118-121.
52. Kang, H A, and Hershey, J W. Effect of initiation factor eIF-5A depletion on protein synthesis and proliferation of Saccharomyces cerevisiae. J Biol. Chem. 1994; 269(6):3934-3940.
53. Chatterjee, I, Gross, S R, Kinzy, T G, and Chen, K Y. Rapid depletion of mutant eukaryotic initiation factor 5A at restrictive temperature reveals connections to actin cytoskeleton and cell cycle progression. Mol Genet Genomics. 2006; 275(3):264-276.
54. Rahman-Roblick, R, Roblick, U J, Hellman, U, Conrotto, P, Liu, T, Becker, S, Hirschberg, D, Jornvall, H, Auer, G, and Wiman, K G. p53 targets identified by protein expression profiling. Proc Natl Acad Sci USA. 2007; 104(13):5401-5406.
55. Bevec, D, Jaksche, H, Oft, M, Wohl, T, Himmelspach, M, Pacher, A, Schebesta, M, Koettnitz, K, Dobrovnik, M, Csonga, R, Lottspeich, F, and Hauber, J Inhibition of HIV-1 replication in lymphocytes by mutants of the Rev cofactor eIF-5A. Science. 1996; 271(5257):1858-1860.
56. Kroncke, K D, Fehsel, K, Suschek, C, and Kolb-Bachofen, V. Inducible nitric oxide synthase-derived nitric oxide in gene regulation, cell death and cell survival. Int Immunopharmacol. 2001; 1(8):1407-1420.
57. Park, M H. The post-translational synthesis of a polyamine-derived amino acid, hypusine, in the eukaryotic translation initiation factor 5A (eIF5A). J Biochem. 2006; 139(2):161-169.
58. Gosslau, A, Jao, D L, Butler, R, Liu, A Y, and Chen, K Y. Thermal killing of human colon cancer cells is associated with the loss of eukaryotic initiation factor 5A. J Cell Physiol. 2009; 219(2):485-493.
59. Takeuchi, K, Nakamura, K, Fujimoto, M, Kaino, S, Kondoh, S, and Okita, K. Heat stress-induced loss of eukaryotic initiation factor 5A (eIF-5A) in a human pancreatic cancer cell line, MIA PaCa-2, analyzed by two-dimensional gel electrophoresis. Electrophoresis. 2002; 23(4):662-669.
60. Elfgang, C, Rosorius, O, Hofer, L, Jaksche, H, Hauber, J, and Bevec, D. Evidence for specific nucleocytoplasmic transport pathways used by leucine-rich nuclear export signals. Proc Natl Acad Sci USA. 1999; 96(11):6229-6234.
61. Hofmann, W, Reichart, B, Ewald, A, Muller, E, Schmitt, I, Stauber, R H, Lottspeich, F, Jockusch, B M, Scheer, U, Hauber, J, and Dabauvalle, M C. Cofactor requirements for nuclear export of Rev response element (RRE)- and constitutive transport element (CTE)-containing retroviral RNAs. An unexpected role for actin. J. Cell Biol. 2001; 152(5):895-910.
62. Schutz, S, Chemnitz, J, Spillner, C, Frohme, M, Hauber, J, and Kehlenbach, R H. Stimulated expression of mRNAs in activated T cells depends on a functional CRM1 nuclear export pathway. J Mol Biol. 2006; 358(4):997-1009.
63. Jang, B C, Sung, S H, Park, J G, Park, J W, Suh, M H, Choi, I H, Yoshida, M, Yoo, S K, and Suh, S I. Leptomycin B, a metabolite of Streptomyces, inhibits the expression of inducible nitric oxide synthase in BV2 microglial cells. Int J Oncol. 2006; 29(6):1509-1515.
64. Zanelli, C F, Maragno, A L, Gregio, A P, Komili, S, Pandolfi, J R, Mestriner, C A, Lustri, W R, and Valentini, S R. eIF5A binds to translational machinery components and affects translation in yeast. Biochem Biophys Res Commun. 2006; 348(4):1358-1366.
65. Bleich, D, Chen, S, Zipser, B, Sun, D, Funk, C D, and Nadler, J L. Resistance to type 1 diabetes induction in 12-lipoxygenase knockout mice. J Clin Invest. 1999; 103(10):1431-1436.
66. McDuffie, M, Maybee, N A, Keller, S R, Stevens, B K, Garmey, J C, Morris, M A, Kropf, E, Rival, C, Ma, K, Carter, J D, Tersey, S A, Nunemaker, C S, and Nadler, J L. Nonobese diabetic (NOD) mice congenic for a targeted deletion of 12/15-lipoxygenase are protected from autoimmune diabetes. Diabetes. 2008; 57(1):199-208.
67. Hyon, S H, Tracey, K J, and Kaufman, D B. Specific inhibition of macrophage-derived proinflammatory cytokine synthesis with a tetravalent guanylhydrazone CNI-1493 accelerates early islet graft function posttransplant. Transplant Proc. 1998; 30(2):409-410.
68. Hohmeier, H E, Mulder, H, Chen, G, Henkel-Rieger, R, Prentki, M, and Newgard, C B. Isolation of INS-1-derived cell lines with robust ATP-sensitive K+ channel-dependent and -independent glucose-stimulated insulin secretion. Diabetes. 2000; 49(3):424-430.
69. Gotoh, M, Ohzato, H, Porter, J, Maki, T, and Monaco, A P. Crucial role of pancreatic ductal collagenase injection for isolation of pancreatic islets. Horm Metab Res Suppl. 1990; 25:10-16.
70. Yang, Z, Chen, M, Ellett, J D, Fialkow, L B, Carter, J D, and Nadler, J L. The novel anti-inflammatory agent lisofylline prevents autoimmune diabetic recurrence after islet transplantation. Transplantation. 2004; 77(1):55-60.
71. Parreiras-E-Silva, L T, Gomes, M D, Oliveira, E B, and Costa-Neto, C M. The N-terminal region of eukaryotic translation initiation factor 5A signals to nuclear localization of the protein. Biochem Biophys Res Commun. 2007; 362(2):393-398.
72. Evans-Molina, C, Robbins, R D, Kono, T, Tersey, S A, Vestermark, G L, Nunemaker, C S, Garmey, J C, Deering, T G, Keller, S R, Maier, B, and Mirmira, R G. PPAR-{gamma} Activation Restores Islet Function in Diabetic Mice Through Reduction of ER Stress and Maintenance of Euchromatin Structure. Mol Cell Biol. 2009; 29(8):2053-2067.
73. Kulkarni, R N, Jhala, U S, Winnay, J N, Krajewski, S, Montminy, M, and Kahn, C R. PDX-1 haploinsufficiency limits the compensatory islet hyperplasia that occurs in response to insulin resistance. J Clin Invest. 2004; 114(6):828-836.
74. Francis, J, Chakrabarti, S K, Garmey, J C, and Mirmira, R G. Pdx-1 links histone H3-Lys-4 methylation to RNA polymerase II Elongation during activation of insulin transcription. J Biol Chem. 2005; 280(43):36244-36253.
75. Sun, B K, Deaton, A M, and Lee, J T. A transient heterochromatic state in Xist preempts X inactivation choice without RNA stabilization. Mol. Cell. 2006; 21(5):617-628.
76. Dignam, J D, Lebovitz, R M, and Roeder, R G. Accurate transcription initiation by RNA polymerase II in a soluble extract from isolated mammalian nuclei. Nucleic Acids Res. 1983; 11(5):1475-1489.
77. Evans-Molina, C, Garmey, J C, Ketchum, R J, Brayman, K, Deng, S, and Mirmira, R G. Glucose regulation of insulin gene transcription and pre-mRNA processing in human islets. Diabetes. 2007; 56:827-835.
78. Iype, T, Francis, J, Garmey, J C, Schisler, J C, Nesher, R, Weir, G C, Becker, T C, Newgard, C B, Griffen, S C, and Mirmira, R G. Mechanism of insulin gene regulation by the pancreatic transcription factor Pdx-1: application of pre-mRNA analysis and chromatin immunoprecipitation to assess formation of functional transcriptional complexes. J Biol. Chem. 2005; 280(17):16798-16807.
79. Jahanshahi, P, Wu, R, Carter, J D, and Nunemaker, C S. Evidence of diminished glucose stimulation and endoplasmic reticulum function in nonoscillatory pancreatic islets. Endocrinology. 2009; 150(2):607-615.

1. An in vivo method for treating a condition or disease comprising:
(a) providing a mammal exhibiting symptoms of said condition or disease; and
(b) treating said mammal with a therapeutically effective amount of an agent capable of blocking or attenuating iNOS translation within said mammal's pancreatic islets, wherein said condition or disease is selected from the group consisting of insulin resistance, an elevated blood glucose level, pre-diabetes, diabetes 1, and diabetes 2.

2. The method of claim 1, wherein said treating involves treating said mammal with a si-RNA.

3. The method of claim 2, wherein said treating involves treating said mammal with a si-RNA which is si-eIF5A.

4. The method of claim 3, wherein the si-eIF5A comprises the nucleotide synthesis 5′-AACGGAAUGACUUCCAGCUGA-3 (SEQ ID NO: 2).

5. The method of claim 1, wherein said treating involves treating said mammal with an inhibitor of deoxyhypusine synthase.

6. The method of claim 5, wherein said treating involves treating said mammal with said inhibitor of deoxyhypusine synthase selected from the group consisting of GC6, GC7, GCB, GC6G, GC7G, GC8G, CNI-1493, and a combination thereof.

7. The method of claim 6, wherein said treating involves treating said mammal with said inhibitor of deoxyhypusine synthase which is GC7.

8. The method of claim 1, wherein said treating involves treating said mammal with an inhibitor of deoxyhypusine hydroxylase.

9. The method of claim 8, wherein said treating involves treating said mammal with an inhibitor of deoxyhypusine hydroxylase which is mimosine.

10. The method of claim 1, wherein said treating involves administering said agent by injection, IV administration, ingestion, dermal application, inhalation, or an osmotic pump.

11. The method of claim 1, wherein said providing a mammal involves providing a human.

12. An in vivo method for controlling a mammal's blood glucose level comprising
(a) providing a mammal exhibiting an elevated blood glucose level.
(b) treating said mammal with an effective amount of an agent capable of reducing iNOS production within said islets, wherein said treating results in said mammal having a blood glucose level lower than said elevated blood glucose level.

13. The method of claim 12, wherein said treating involves treating said mammal with a si-RNA.

14. The method of claim 13, wherein said si-RNA is si-eIF5A.

15. The method of claim 14, wherein the si-eIF5A comprises the nucleotide synthesis 5′-AACGGAAUGACUUCCAGCUGA-3 (SEQ ID NO: 2).

16. The method of claim 12, wherein said treating involves treating said mammal with an inhibitor of deoxyhypusine synthase.

17. The method of claim 16, wherein said inhibitor of deoxyhypusine synthase is selected from the group consisting of GC6, GC7, GCB, GC6G, GC7G, GC8G, CNI-1493, and a combination thereof.

18. The method of claim 17, wherein said inhibitor of deoxyhypusine synthase is GC7.

19. The method of claim 12, wherein said treating involves treating said mammal with an inhibitor of deoxyhypusine hydroxylase.

20. The method of claim 19, wherein said inhibitor of deoxyhypusine hydroxylase is an inhibitor of deoxyhypusine hydroxylase which is mimosine.

21. The method of claim 12, wherein said treating involves administering said agent by injection, IV administration, ingestion, dermal application, inhalation, or an osmotic pump.

22. The method of claim 12, wherein said providing involves providing a human.

23. The method of claim 12, wherein said providing involves providing a mammal suffering from diabetes.

24. The method of claim 23, wherein said providing involves providing a mammal suffering from type 1 diabetes and said treating results in said mammal exhibiting a blood glucose level normal for said mammal.

25. The method of claim 23, wherein said providing involves providing a mammal suffering from type 2 diabetes and said treating results in said mammal exhibiting a blood glucose level normal for said mammal.

26. An in vivo method for treating a condition or disease comprising:
(a) providing a mammal exhibiting symptoms of said condition or disease; and
(b) treating said mammal with a therapeutically effective amount of an agent capable of inhibiting hypusination of eIF5A within said mammal's pancreatic islets, wherein said condition or disease is selected from the group consisting of insulin resistance, an elevated blood glucose level, pre-diabetes, diabetes 1, and diabetes 2.

27. The method of claim 26, wherein said agent capable of inhibiting hypusination of eIF5A inhibits deoxyhypusine synthase (DHS).

28. The method of claim 26, wherein said agent capable of inhibiting hypusination of eIF5A inhibits deoxyhypusine hydroxylase (DOHH).

29. The method of claim 27 wherein said agent capable of inhibiting hypusination of eIF5A inhibits deoxyhypusine synthase (DHS) is selected from the group consisting of GC6, GC7, GC8, GC6G, GC7G, GC8G, CNI-1493, and a combination thereof.

30. The method of claim 29, wherein said agent capable of inhibiting deoxyhypusine synthase (DHS) is GC7.

31. The method of claim 28 wherein said agent capable of inhibiting deoxyhypusine hydroxylase (DOHH) is mimosine.

32. A composition for treating a condition or disease comprising an agent capable of inhibiting iNOS translation within a pancreatic cell included in a pharmaceutically acceptable carrier, wherein: (a) said agent is selected from the group consisting of GC6, GC7, GC8, GC6G, GC7G, GC8G, CNI-1493, and a combination thereof; (b) said condition or disease is selected from the group consisting of insulin resistance, an elevated blood glucose level, pre-diabetes, diabetes 1, and diabetes 2; and (c) said agent is included in said composition at a concentration ranging from about 0.1 μM to about 200 μM.

33. The composition of claim 32, wherein said agent selected is GC7.

34. A composition for treating a condition or disease comprising an agent capable of inhibiting iNOS translation within a pancreatic cell included in a pharmaceutically acceptable carrier, wherein: (a) said agent is si-eIF5A and (b) said condition or disease is selected from the group consisting of insulin resistance, an elevated blood glucose level, pre-diabetes, diabetes 1, and diabetes 2.

35. The composition of claim 34, wherein said si-eIF5A includes the nucleotide sequence 5′-AACGGAAUGACUUCCAGCUGA-3 (SEQ ID NO: 2).


SEQUENCE LISTING (2 sequences)

SEQ ID NO: 1 - RNA, 21 nt, Artificial Sequence
  Synthetic oligonucleotide
aaagucgaccuucaguaagga

SEQ ID NO: 2 - RNA, 21 nt, Artificial Sequence
  Synthetic oligonucleotide
aacggaaugacuuccagcuga